SEQUENCE LISTING (6 sequences)

SEQ ID NO: 1 - DNA, 22 bp, Artificial Sequence
  sgRNA #1
tgctgttctgctgccccctcgg

SEQ ID NO: 2 - DNA, 23 bp, Artificial Sequence
  sgRNA #2
ataggcgctgtgggggtccaggg

SEQ ID NO: 3 - DNA, 59 bp, Artificial Sequence
  Part of JAK 3 CDS Exon 2
tctgctgccccctcggggccctggacccccacagcgcctatccttctcctttggggacc

SEQ ID NO: 4 - DNA, 60 bp, Artificial Sequence
  Part of mutated JAK 3 CDS Exon 2 (+1bp)
tctgctgccccctcggggccctggaacccccacagcgcctatccttctcctttggggacc

SEQ ID NO: 5 - DNA, 58 bp, Artificial Sequence
  Part of mutated JAK 3 CDS Exon 2 (-1bp)
tctgctgccccctcggggccctggaccccacagcgcctatccttctcctttggggacc

SEQ ID NO: 6 - DNA, 42 bp, Artificial Sequence
  Part of mutated JAK 3 CDS Exon 2 (-17bp)
tctgctgccccctcggggccctatccttctcctttggggacc

(19) United States
(12) Patent Application Publication
(10) Pub. No.: US 2024/0008460 A1
(43) Pub. Date: Jan. 11, 2024

(54) JAK3 GENE-MUTATED, SEVERE COMBINED IMMUNODEFICIENCY ANIMAL MODEL AND CONSTRUCTION METHOD THEREFOR

(71) Applicant: KOREA RESEARCH INSTITUTE OF BIOSCIENCE AND BIOTECHNOLOGY, Daejeon (KR)

(72) Inventors: Sun Uk KIM, Daejeon (KR); Young Ho PARK, Daejeon (KR); Bo Woong SIM, Daejeon (KR); Kyu Tae CHANG, Daejeon (KR); Seung Hwan LEE, Daejeon (KR); Bong Seok SONG, Daejeon (KR); Pil Soo JEONG, Daejeon (KR); Hae Jun YANG, Daejeon (KR); Sang Rae LEE, Daejeon (KR); Yeung Bae JIN, Daejeon (KR); Kang Jin JEONG, Daejeon (KR)

(73) Assignee: KOREA RESEARCH INSTITUTE OF BIOSCIENCE AND BIOTECHNOLOGY, Daejeon (KR)

(21) Appl. No.: 18/034,995

(22) PCT Filed: Oct. 15, 2021
(86) PCT No.: PCT/KR2021/014391

§ 371 Date: Jul. 3, 2023

(30) Foreign Application Priority Data
Nov. 3, 2020 (KR) 10-2020-0145305

(51) Int. Cl. A01K 67/027; C12N 9/22; C12N 15/11; C12N 15/90

(52) U.S. Cl. CPC A01K 67/0276; C12N 9/22; C12N 15/11; C12N 15/907; A01K 2227/108; A01K 2217/075; A01K 2217/054; A01K 2267/0387; C12N 2310/20

(57) ABSTRACT

The present disclosure relates to a JAK3 gene-mutated severe combined immunodeficiency animal model and a method of constructing the same. In the JAK3 gene-mutated severe combined immunodeficiency animal model of the present disclosure, the JAK3 gene is specifically deficient, the expression of cytokines is regulated by controlling the number and activity of macrophages, and the thymus, lymphocytes, and Peyer's patches, which are observed in conventional severe combined immunodeficiency animal models, particularly mini-pigs, are completely lacking. In addition, the animal model of the present disclosure can be used as a treatment model for JAK3 SCID patients, as similar phenotypes are observed in patients with human severe combined immunodeficiency caused by a JAK3 gene mutation, and can be used for artificial blood development or xenotransplantation.

### TECHNICAL FIELD

The present disclosure relates to a severe combined immunodeficiency animal model with a JAK3 gene mutation and a method of constructing the same.

The present disclosure was made with the support of the Korean government in accordance with the Ministry of Science and ICT's task number KGM4252021, “Development Project of General-Purpose/Personalized Artificial Blood Using Mini-Pig Resources.”

### BACKGROUND ART

Severe combined immunodeficiency (SCID) refers to a genetic disorder characterized by the developmental or functional abnormalities of major immune cells such as T cells, B cells, and natural killer (NK) cells. Mutations may occur in at least 12 genes, including interleukin-2 receptor gamma (IL2rg), recombination activating gene 1/2 (RAG1/2), and Janus kinase 3 (JAK3). Despite other causes, most patients with SCID have small thymuses or lack thymocytes, lymph nodes, or tonsils. Although many approaches to treat SCID (e.g., stem cell and bone marrow transplantation and enzyme replacement therapy) are being developed, patients with SCID remain at severe risk to date.

Severe combined immunodeficiency animal models (IL2rg, JAK3, RAG1, and RAG2) have been developed in mice, but it is known that they show significant differences in the immune system from humans in basic and clinical studies. Therefore, an attempt was made to develop RAG1, RAG2, IL2rg, and RAG1/3, RAG2/IL2rg knock-out pigs in an attempt to develop an SCID animal model in pigs with similar immune characteristics to humans.

JAK3 is a tyrosine kinase related to the Janus family of kinases, and is known to play an important role in the immune system as a receptor subunit for various cytokine receptors, including interleukin (IL) 2, 4, 7, 9, 15 and 21. It is also known to cause autosomal SCID and account for about 7% of SCID in human patients. In immune cell dysfunction, T−, B+, and NK− phenotypes appear because the common gamma chain (γc) of IL2rg is linked to components in the same signal transduction pathway.

However, even in the case of mice, despite similar phenotypes such as interference with the IL-2 signaling pathway related to T cell development, severe damage to thymus development involved in T cell maturation, and absence of ileal Peyer's patches and lymph nodes, some researchers have confirmed that JAK3 knock-out mice were different from IL2rg knock-out mice. In particular, JAK3 knock-out mice show more severe thymic hypoplasia compared to IL2rg knock-out mice, and thymic bodies are detected in the thymic medulla of IL2rg knock-out mice, but not in JAK3 knock-out mice.

Therefore, this research team used the genetic scissors (CRISPR/Cas9) system to delete the JAK3 gene in mini-pig somatic cells, and then produced mini-pigs in which the JAK3 gene was knocked out, using the transformed somatic cells through somatic cell cloning, and confirmed the immunological characteristics thereof.

### DETAILED DESCRIPTION OF THE DISCLOSURE

### Technical Problem

One object of the present disclosure is to provide a recombinant expression vector including a nucleotide sequence encoding a gRNA (guide RNA) that hybridizes to a DNA encoding a Janus kinase 3 (JAK3) gene, a nucleotide sequence encoding a Cas9 protein, and a promoter operably linked to the nucleotide sequence.

Another object of the present disclosure is to provide a transgenic cell line for constructing a severe combined immunodeficiency animal model into which the recombinant expression vector is introduced.

Another object of the present disclosure is to provide a method of constructing a severe combined immunodeficiency animal model, the method including forming a nuclear transfer embryo by transplanting the transgenic cell line into an enucleated egg obtained from an animal other than a human; and transplanting the nuclear transfer embryo into a fallopian tube of a surrogate mother, which is an animal other than a human.

Another object of the present disclosure is to provide a severe combined immunodeficiency animal model with a Janus kinase 3 (JAK3) gene mutation.

### Technical Solution to Problem

One aspect of the present disclosure provides a recombinant expression vector including: a nucleotide sequence encoding a guide RNA (gRNA) that hybridizes to a DNA encoding a Janus kinase 3 (JAK3) gene; a nucleotide sequence encoding a Cas9 protein; and a promoter operably linked to the nucleotide sequence.

In an embodiment of the present disclosure, the gRNA consists of the nucleotide sequence of SEQ ID NO: 1 or SEQ ID NO: 2.

Another aspect of the present disclosure provides a transgenic cell line for constructing a severe combined immunodeficiency animal model into which the recombinant expression vector is introduced.

Another aspect of the present disclosure provides a method of constructing a severe combined immunodeficiency animal model, the method including: forming a nuclear transfer embryo by transplanting the transgenic cell line into an enucleated egg obtained from an animal other than a human; and transplanting the nuclear transfer embryo into the fallopian tube of a surrogate mother, which is an animal other than a human.

In an embodiment of the present disclosure, the animal may be a mini-pig.

In another embodiment of the present disclosure, the severe combined immunodeficiency may be caused by Janus kinase 3 (JAK3) knock-out.

In an embodiment of the present disclosure, the knock-out may be generated by mutating a nucleotide sequence corresponding to SEQ ID NO: 3 to any one nucleotide sequence selected from the group consisting of SEQ ID NO: 4 to SEQ ID NO: 6.

Another aspect of the present disclosure provides a severe combined immunodeficiency animal model with a Janus kinase 3 (JAK3) gene mutation.

In an embodiment of the present disclosure, the animal model may be an animal model in which the JAK3 gene is knocked out.

In an embodiment of the present disclosure, the knock-out may be generated by mutating a nucleotide sequence corresponding to SEQ ID NO: 3 to any one nucleotide sequence selected from the group consisting of SEQ ID NO: 4 to SEQ ID NO: 6.

In an embodiment of the present disclosure, the animal may be a mini-pig.

In an embodiment of the present disclosure, the animal may be used for artificial blood development, xenotransplantation, or severe immunodeficiency disease animal models.

### Advantageous Effects of Disclosure

The present disclosure can provide a recombinant expression vector capable of providing an animal model in which only the JAK3 gene is specifically knocked out, a cell line including the same, and a method of constructing a severe combined immunodeficiency animal model, and can also provide a severe combined immunodeficiency animal model constructed thereby. In the animal model of the present disclosure, the JAK3 gene is specifically deficient, the expression of cytokines is regulated by controlling the number and activity of macrophages, and the thymus, lymphocytes, and Peyer's patches observed in conventional severe combined immunodeficiency animal models, especially mini pigs, are completely lacking. In addition, the animal model of the present disclosure can be used as a treatment model for JAK3 SCID patients, since a similar phenotype appearing in patients with human severe combined immunodeficiency caused by a JAK3 gene mutation is observed, and can be used for artificial blood development or xenotransplantation.

### BRIEF DESCRIPTION OF DRAWINGS

FIG. 1 is a view illustrating the guide RNA sequence and locus of CRISPR/Cas9 gene scissors that hybridize to JAK3 exon 2 according to Experimental Example 1-1 of the present application.

FIG. 2 is a view illustrating the structures of a porcine JAK3 reporter vector, a JAK3 gRNA vector, and a Cas9 vector of JAK3 knock-out gene scissors according to Experimental Example 1-2-1 of the present application.

FIG. 3 is a set of images verifying the introduction and operation of a JAK3 knock-out gene scissors expression vector system according to Experimental Example 1-2-2 of the present application.

FIG. 4 illustrates the flow cytometry results of verifying a JAK3 knock-out gene scissors expression vector system according to Experimental Example 1-2-3 of the present application.

FIG. 5 illustrates the T7E1 analysis results of verifying a JAK3 knock-out gene scissors expression vector system according to Experimental Example 1-2-4 of the present application.

FIG. 6 is a view illustrating a method of Experimental Example 1-3 of the present application.

FIG. 7 is a view illustrating mutations by the JAK3 knock-out gene expression vector system according to Experimental Examples 1-3 of the present application.

FIG. 8 illustrates the amino acid sequence analysis results of verifying a JAK3 knock-out gene scissors expression vector system according to Experimental Example 1-4 of the present application.

FIGS. 9 to 11 illustrate the karyotyping results of verifying a JAK3 knock-out gene scissors expression vector system according to Experimental Example 1-5 of the present application.

FIG. 12 is a view illustrating a process of producing JAK3 knock-out transgenic cloned mini-pigs according to Example 1 of the present application.

FIGS. 13 and 14 are images showing a process of producing JAK3 knock-out transgenic cloned mini-pigs and the produced mini-pigs, according to Example 1 of the present application.

FIG. 15 is a view confirming JAK3 gene knock-out in JAK3 knock-out transgenic cloned mini-pigs according to Experimental Example 2-1 of the present application.

FIG. 16 illustrates the results according to Experimental Example 2-2-1 of the present application, particularly the results of RT-PCR analysis of mRNA of JAK3 knock-out transgenic cloned mini-pigs.

FIG. 17 illustrates the results according to Experimental Example 2-2-2 of the present application, particularly the results of western blot analysis of mRNA of JAK3 knock-out transgenic cloned mini-pigs.

FIGS. 18 and 19 illustrate the results according to Experimental Example 3-1 of the present application, particularly the results of confirming the thymus, spleen, and submandibular or mesenteric lymph nodes of JAK3 knock-out transgenic cloned mini-pigs.

FIG. 20 illustrates the results according to Experimental Example 3-2 of the present application, particularly the results of confirming tissue formation in the thymus and spleen of JAK3 knock-out transgenic cloned mini-pigs by immunohistochemical staining.

FIGS. 21 and 22 illustrate the results according to Experimental Example 3-3 of the present application, particularly the results of immunohistochemical staining and flow cytometry of spleen tissue.

FIGS. 23 and 24 illustrate the results according to Experimental Example 3-5 of the present application, particularly the results of confirming the formation of immune cells by a V(D)J recombination detection method.

FIG. 25 illustrates the results according to Experimental Example 4-1 of the present application, particularly the results of survival rate analysis of JAK3 knock-out transgenic cloned mini-pigs.

FIG. 26 illustrates the results according to Experimental Example 4-2 of the present application, particularly the results of confirming cancer cell growth in JAK3 knock-out transgenic cloned mini-pigs.

FIGS. 27 and 28 illustrate the results according to Experimental Example 4-3 of the present application, particularly the results of confirming the introduction of immune cells into cancer cells and tissue transplanted into JAK3 knock-out transgenic cloned mini-pigs by immunohistochemical staining and RT-PCR.

FIG. 29 illustrates the results according to Experimental Example 5-1 of the present application, particularly the results of blood analysis of JAK3 knock-out transgenic cloned min-pigs.

FIGS. 30 and 31 illustrate the results according to Experimental Example 5-2 of the present application, particularly the results of confirming the expression of macrophage-related genes in the kidney tissue of JAK3 knock-out transgenic cloned mini-pigs.

FIGS. 32 and 33 illustrate the results according to Experimental Example 5-3 of the present application, particularly the results of confirming the expression of CD68 among macrophage-related genes in the kidney tissue of JAK3 knock-out transgenic cloned mini-pigs.

FIGS. 34 to 36 illustrate the results according to Experimental Example 5-4 of the present application, particularly the results of confirming the expression of macrophage polarization-related genes (M1, M2) and the expression of cytokines, in the kidney tissue of JAK3 knock-out transgenic cloned mini-pigs.

FIGS. 37 to 44 illustrate the results according to Experimental Example 6-1 of the present application, particularly the results of comparing and confirming lung/visceral abnormalities in JAK3 knock-out (KO) transgenic cloned mini-pigs.

FIG. 45 illustrates the results according to Experimental Example 6-2 of the present application, particularly the results of comparing and confirming visceral abnormalities in JAK3 knock-out (KO) transgenic cloned mini-pigs.

### BEST MODE

One aspect of the present disclosure provides a recombinant expression vector including: a nucleotide sequence encoding a guide RNA (gRNA) that hybridizes to a DNA encoding a Janus kinase 3 (JAK3) gene; a nucleotide sequence encoding a Cas9 protein; and a promoter operably linked to the nucleotide sequence.

In the present specification, there is provided a method of producing a transgenic animal having the phenotype of severe combined immunodeficiency, by knocking out the JAK3 gene by using JAK3-specific gene scissors using a CRISPR/Cas9 system, and applying a transgenic somatic cell cloning technique.

The term “CRISPR/Cas9 system” as used herein refers to third-generation gene scissors consisting of a Cas9 protein and a guide RNA, and refers to an artificial restriction enzyme designed to cleave a desired gene sequence using a Clustered Regularly Interspaced Short Palindromic Repeats (CRISPR) system known as a microbial immune system.

The term “Cas9 protein” as used herein refers to an essential protein element in the CRISPR/Cas9 system, which may act as active endonuclease or nickase by forming a complex with two RNAs called CRISPR RNA (crRNA) and trans-activating crRNA (tracrRNA). The Cas9 protein may be derived from Staphylococcus sp., Streptococcus sp., Neisseria sp., Pasteurella sp., Francisella sp., or Campylobacter sp.

The term “guide RNA (gRNA)” as used herein refers to an RNA specific to a target DNA, and gRNA may form a complex with the Cas9 protein, and may direct the Cas9 protein to a target DNA. gRNA may be crRNA and tracrRNA, or may be a single guided RNA (sgRNA) in which crRNA and tracrRNA are linked to each other.

When a cell is transformed with the recombinant expression vector of the present disclosure, a gRNA fragment may be delivered into the cell, and the delivered gRNA fragment may recognize the JAK3 gene. The recombinant expression vector may further include tracrRNA. Therefore, when a cell is transformed with the recombinant expression vector, a gRNA fragment and a tracrRNA fragment or an sgRNA in which crRNA and tracrRNA are linked to each other may be delivered into the cell, and the delivered tracrRNA fragment or moiety may serve to form a complex or linkage structure with a crRNA fragment or moiety, thus forming a structure that may be recognized by the Cas9 protein.

The term “Janus kinase 3 (JAK3)” as used herein refers to a member of the Janus family of protein kinases. Other members of this family are expressed by virtually all tissues, but JAK3 expression is restricted to hematopoietic cells. This is consistent with its essential role in signaling through the receptors for IL-2, IL-4, IL-7, IL-9 and IL-15 by non-covalent linkage of JAK3 with the gamma chain common to multichain receptors. XSCID patient populations have been found to have genetic defects for common gamma chains or severely reduced levels of the JAK3 protein, suggesting that immune suppression must result from blockade of signaling through the JAK3 pathway. JAK3 is known to play an important role in B and T lymphocyte maturation, as well as being constitutively required for maintaining T cell function.

The term “knock-out” as used herein means modifying or removing a specific gene in the nucleotide sequence so that the gene cannot be expressed.

The term “recombinant expression vector” as used herein refers to a recombinant DNA molecule containing a desired coding sequence and appropriate nucleic acid sequences essential for the expression of an operably linked coding sequence in a particular host organism. Promoters, enhancers, termination signals, and polyadenylation signals, which may be used in eukaryotic cells, are known.

The term “operably linked” as used herein refers to a functional linkage between a gene expression control sequence and another nucleotide sequence. The gene expression control sequence may be at least one selected from the group consisting of a replication origin, a promoter, and a transcription termination sequence (terminator). The transcription termination sequence may be a polyadenylation sequence (pA), and the origin of replication may be, but is not limited to, an f1 replication origin, an SV40 replication origin, a pMB1 replication origin, an adeno replication origin, an AAV replication origin, or a BBV replication origin.

The term “promoter” as used herein means a region of DNA upstream from a structural gene, and refers to a DNA molecule to which RNA polymerase binds to initiate transcription.

The promoter according to an embodiment of the present disclosure is one of the transcription control sequences which regulate the transcription initiation of a specific gene, and may be a polynucleotide fragment of about 100 bp to about 2,500 bp in length. The promoter can be used without limitation as long as it can regulate transcription initiation in cells, for example, eukaryotic cells (e.g., plant cells or animal cells (e.g., mammalian cells such as human or mouse cells)). For example, the promoter may be selected from the group consisting of a cytomegalovirus (CMV) promoter (e.g., human or mouse CMV immediate-early promoter), U6 promoter, elongation factor 1-α (EF1-alpha) promoter, EF1-alpha short (EFS) promoter, SV40 promoter, adenovirus promoter (major late promoter), pLλ promoter, trp promoter, lac promoter, tac promoter, T7 promoter, vaccinia virus 7.5K promoter, HSV tk promoter, SV40E1 promoter, respiratory syncytial virus (RSV) promoter, metallothionin promoter, β-actin promoter, ubiquitin C promoter, human interleukin-2 (IL-2) gene promoter, human lymphotoxin gene promoter, and human granulocyte-macrophage colony stimulating factor (GM-CSF) gene promoter, but is not limited thereto.

The recombinant expression vector according to an embodiment of the present disclosure may be selected from the group consisting of plasmid vectors, cosmid vectors, and viral vectors such as bacteriophage vectors, adenovirus vectors, retroviral vectors, and adeno-associated viral vectors. A vector that may be used as the recombinant expression vector may be constructed on the basis of, but not being limited to, a plasmid (e.g., pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, and pUC19), a phage (e.g., λgt4λB, λ-Charon, λΔz1, and M13), a viral vector (e.g., an adeno-associated viral (AAV) vector), or the like, which are used in the art.

The recombinant expression vector of the present disclosure may further include at least one selectable marker. The marker is generally a nucleic acid sequence having the property capable of being selected by a chemical method, and includes any gene capable of distinguishing a transfected cell from a non-transfected cell. Examples of the marker include, but are not limited to, genes resistant to herbicides such as glyphosate, glufosinate ammonium or phosphinothricin, and genes resistant to antibiotics such as ampicillin, kanamycin, G418, bleomycin, hygromycin or chloramphenicol.

The recombinant expression vector of the present disclosure may be constructed using a gene recombination technique well known in the art, and site-specific DNA cleavage and ligation may be performed using enzymes generally known in the art.

In an embodiment of the present disclosure, the gRNA may consist of the nucleotide sequence of SEQ ID NO: 1 or SEQ ID NO: 2.

By using the gRNA consisting of the nucleotide sequence of SEQ ID NO: 1 or SEQ ID NO: 2, a change in the amino acid codon encoding JAK3 and/or the generation of a premature stop codon may be efficiently induced. The gRNA has the effect of causing a specific deletion of only the JAK3 gene as a result of off-target analysis.

Another aspect of the present disclosure provides a transgenic cell line for constructing a severe combined immunodeficiency animal model, into which the recombinant expression vector is introduced, and in particular, provides a transgenic cell line for constructing a severe combined immunodeficiency animal model, into which a recombinant expression vector, including: a nucleotide sequence encoding a guide RNA (gRNA) that hybridizes to a DNA encoding a Janus kinase 3 (JAK3) gene; a nucleotide sequence encoding a Cas9 protein; and a promoter operably linked to the nucleotide sequence, is introduced.

The term “severe combined immunodeficiency (SCID)” as used herein refers to a condition in which an effective immune response is lacking, and particularly, SCID may be caused by deficiency in the development and function of T, B and NK cells.

To produce a transgenic cell line into which the recombinant expression vector according to an embodiment of the present disclosure is introduced, a method known in the art for introducing a nucleic acid molecule into an organism, a cell, a tissue or an organ may be used, and as known in the art, a suitable standard technique selected depending on the host cell may be performed. Examples of this method include, but are not limited to, electroporation, calcium phosphate (CaPO4) precipitation, calcium chloride (CaCl2))) precipitation, microinjection, a polyethylene glycol (PEG) method, a DEAE-dextran method, a cationic liposome method, and a lithium acetate-DMSO method.

The type of a cell to be used as the transgenic cell line may be animal cells or cells derived from animal cells, preferably somatic cells derived from mammals, and most preferably, somatic cells derived from pigs or mini-pigs. When somatic cells derived from mini-pigs are used as the transgenic cell line, the problem of mini-pig death immediately after birth may be overcome, and severe combined immunodeficiency may be induced.

Another aspect of the present disclosure provides a method of constructing a severe combined immunodeficiency animal model, including the steps of: forming a nuclear transfer embryo by transplanting the transgenic cell line into an enucleated egg obtained from an animal other than a human; and transplanting the nuclear transfer embryo into the fallopian tube of a surrogate mother, which is an animal other than a human, and particularly, provides a method of constructing a severe combined immunodeficiency animal model, including a step of transplanting, into the fallopian tube of a surrogate mother, which is an animal other than a human, a transgenic cell line for constructing a severe combined immunodeficiency, into which a recombinant expression vector is introduced, the recombinant expression vector including: a nucleotide sequence encoding a guide RNA (gRNA) that hybridizes to a DNA encoding a Janus kinase 3 (JAK3) gene; a nucleotide sequence encoding a Cas9 protein; and a promoter operably linked to the nucleotide sequence.

The method of constructing a severe combined immunodeficiency animal model may be performed by somatic cell nuclear transfer (SCNT). “Somatic cell nuclear transfer” is a gene manipulation technique capable of producing offspring without passing through meiotic and haploid germ cells, which generally occur in the reproductive process, and is a method including producing a fertilized egg by transplanting a polyploid somatic cell of an adult into an enucleated egg, and generating a new individual by transplanting the fertilized egg into a living body.

The term “nuclear transfer embryo” as used herein refers to an egg produced by insertion or fusion of a nuclear donor cell, and the term “fusion” as used herein refers to a combination of a nuclear donor cell and a lipid membrane portion of an egg. For example, the lipid membrane may be the plasma membrane or nuclear membrane of a cell. Fusion may occur upon application of an electrical stimulus, when a nuclear donor cell and an egg are placed adjacent to each other or when a nuclear donor cell is placed in the perivitelline space of a recipient egg. The transgenic cell line is a nuclear donor cell and refers to a cell or a nucleus of the cell which transfers the nucleus into an oocyte functioning as a nuclear recipient. The oocyte preferably refers to a mature oocyte which has reached metaphase II of meiosis, and may be preferably a porcine oocyte.

According to an embodiment of the present disclosure, the animal may be a mini-pig.

Pigs have been recognized to be anatomically and physiologically similar to humans and have been already used in studies on the pathological mechanisms and treatment of various diseases. Particularly, pigs have been recognized as an economic animal for a long time, and thus the use thereof can avoid ethical problems, unlike when other medium/large animals are used as disease models. In addition, since a stable breeding system for pigs is already established, pigs are advantageously easy to maintain and control during the development of an experimental animal model.

In an embodiment of the present disclosure, the severe combined immunodeficiency may be caused by Janus kinase 3 (JAK3) knock-out.

The severe combined immunodeficiency animal model can be constructed by JAK3 knock-out through substitution or deletion of a part of nucleotides constituting the JAK3 gene, or insertion of some nucleotides.

The term “knock-out” as used herein means modifying or removing a specific gene in the nucleotide sequence so that the gene cannot be expressed.

In an embodiment of the present disclosure, the knock-out may be generated by mutating a nucleotide sequence corresponding to SEQ ID NO: 3 to any one nucleotide sequence selected from the group consisting of SEQ ID NO: 4 to SEQ ID NO: 6.

Specifically, SEQ ID NO: 3 is part of the sequence of exon 2 (EXON 2) of JAK 3, and includes a portion that hybridizes with a gRNA consisting of the nucleotide sequence of SEQ ID NO: 1 or SEQ ID NO: 2 as described above. In addition, the nucleotide sequences of SEQ ID NO: 4 to SEQ ID NO: 6 are sequences generated by mutation through a gRNA consisting of the nucleotide sequence of SEQ ID NO: 1 or SEQ ID NO: 2 and genetic scissors (Cas9), and have a 1-bp increase/decrease or a 17-bp deletion. More specifically, SEQ ID NO: 4 is a nucleotide sequence in which ‘A’ is inserted at position 25 of SEQ ID NO: 3. SEQ ID NO: 5 is a nucleotide sequence in which ‘C,’ which is a nucleotide at position 30 of SEQ ID NO: 3, is deleted. SEQ ID NO: 6 is a nucleotide sequence in which

′GGACCCCCACAGCGCCT,′

which are nucleotides at position 23 to position 39 of SEQ ID NO: 3, are deleted.

Another aspect of the present disclosure provides a severe combined immunodeficiency animal model with a Janus kinase 3 (JAK3) gene mutation.

The term “animal model” as used herein refers to an animal having a disease very similar to a human disease. The reason why disease model animals have significance in studies on human diseases is because of the physiological or genetic similarities between humans and animals. In disease studies, biomedical disease model animals may provide data for studies on various causes of diseases and the onset processes and diagnosis of diseases. Thus, through studies on disease model animals, it is possible to find out genes related to diseases and to understand the interactions between genes, and through examination of the actual efficacy and toxicity of developed new drug candidates, it is possible to obtain basic data for determining the possibility of practical use of the new drug candidates.

In the severe combined immunodeficiency animal model of the present disclosure, redundant description with the foregoing description may be construed as having the same meaning as that described above.

The term “mutation” as used herein refers to a state in which a genotype has changed with a change in genetic information due to a change in the nucleotide sequence of a gene, and examples of this mutation may include point mutations, deletion mutations, insertion mutations, missense mutations, and nonsense mutations. The present disclosure provides a severe combined immunodeficiency animal model in which the expression of the JAK3 gene is reduced due to a mutation in the JAK3 gene by a CRISPR/Cas9 system.

In an embodiment of the present disclosure, the animal model may be an animal model in which the JAK3 gene is knocked out.

In an embodiment of the present disclosure, the knock-out may be caused by mutating a nucleotide sequence corresponding to SEQ ID NO: 3 to any one nucleotide sequence selected from the group consisting of SEQ ID NO: 4 to SEQ ID NO: 6.

In an embodiment of the present disclosure, the animal may be a mini-pig.

In an embodiment of the present disclosure, the animal may be used for artificial blood development, xenotransplantation, or severe combined immunodeficiency animal models.

The severe combined immunodeficiency animal model of the present disclosure, particularly a mini-pig, has characteristics similar to those of the human immune system compared to conventional severe combined immunodeficiency mice. As a result, a phenotype similar to that of severe human immunodeficiency patients is observed in the animal model, and the animal model may be used as a model for human JAK3 SCID research. In addition, in the severe combined immunodeficiency animal model of the present disclosure, the expression of cytokines is regulated by controlling the number and activity of macrophages, and the thymus, lymphocytes, and Peyer's patches are completely lacking, compared to RAG2 knock-out mini-pigs, which are conventional severe combined immunodeficiency mini-pigs. Thus, the animal model is suitable as a severe combined immunodeficiency model.

### MODE OF DISCLOSURE

Hereinafter, one or more specific embodiments will be described in further detail through the following examples. However, these examples are intended to illustrate one or more specific examples and are not intended to limit the scope of the present disclosure.

### Experimental Example 1: Construction of JAK3 Knock-out Mini-Pig Somatic Cells

gRNAs capable of hybridizing to the JAK3 gene of mini-pigs were selected, and JAK3 knock-out mini-pig somatic cells with the gRNAs applied thereto were constructed, to confirm the efficacy.

### Experimental Example 1-1. Selection of gRNAs that Hybridize to JAK3 Gene

gRNAs binding to gene scissors (CRISPR/Cas9) were designed by targeting exon 2 having a coding region related to protein expression among four exons of the porcine JAK3 gene.

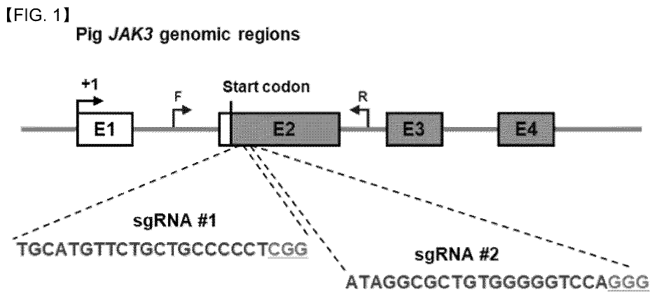

Specifically, as confirmed in FIG. 1, gRNAs (sgRNA #1, sgRNA #2) that hybridize to the gene of exon 2 among the four exons of the porcine JAK3 gene were selected/produced, and these were SEQ ID NO: 1 and SEQ ID NO: 2.

### Experimental Example 1-2. Evaluation of Efficacy of gRNAs Hybridizing to JAK3 Gene

A gene scissors (CRISPR/Cas9) system using each of the gRNA selected/produced in Experimental Example 1-1 was introduced into mini-pig somatic cells, and then the efficacy of the gRNAs was confirmed.

### Experimental Example 1-2-1. JAK3 Knock-out Gene Scissors Expression Vector System

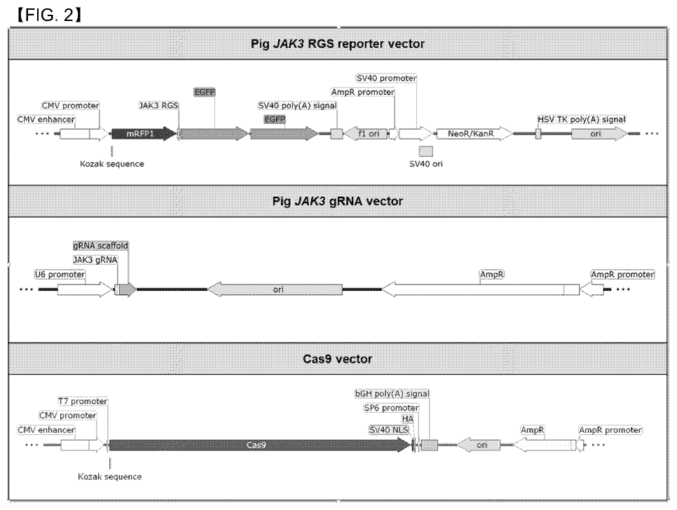

The JAK3 knock-out gene scissors system was constructed using the Surrogate Reporter system of a total of three vectors. Specifically, a reporter vector was constructed in which a monomeric red fluorescent protein 1 coding sequence is located upstream of the gRNA-hybridizing JAK3 nucleotide sequence and an enhanced green fluorescent protein (EGFP) coding sequence is located downstream of the sgRNA-hybridizing JAK3 nucleotide sequence. In addition, a gRNA vector in which a sequence encoding each of the gRNAs selected in Experimental Example 1-1 is located was constructed, and a Cas9 vector was constructed by inserting a sequence encoding a Cas9 protein for recognizing a gRNA, which is expressed from the gRNA vector and bound to the gRNA-hybridizing JAK3 nucleotide sequence, and cleaving the target sequence of JAK3 (sgRNA #1 and sgRNA #2 for each gRNA, see FIG. 2).

For the JAK3 gene target gRNA vector, to achieve the highest knock-out efficiency in the JAK3 coding region using CRISPR, four target specific sites with higher out-of-frame scores were created through published design tools (http://crispr.mit.edu; https://crispr.cos.uni-heidelberg.de/; http://www.rgenome.net/cas-designer/), and the designed Cas9, gRNA and RGS reporter vectors were synthesized by and purchased from ToolGen, Inc. Seoul, Korea.

Since mRFP (red fluorescence) is expressed in cells into which the gene scissors system of the present disclosure has been introduced, it can be verified whether the gene scissors system of the present disclosure is introduced into cells. Meanwhile, when the JAK3 target sequence included in the reporter vector is cleaved by the Cas9 protein expressed from the Cas9 vector, EGFP (green fluorescence) is expressed in cells into which the gene scissors system of the present disclosure has been introduced. Thus, by confirming whether green fluorescence is emitted, it is possible to verify whether or not the gene scissors system of the present disclosure operates normally.

### Experimental Example 1-2-2. Verification of JAK3 Knock-out Gene Scissors Expression Vector System-Fluorescence Microscopy

To product JAK3 knock-out transgenic cloned mini-pigs, the JAK3 knock-out gene scissors expression vector system constructed in Experimental Example 1-2-1 was introduced into a normal mini-pig donor cell line for somatic cell cloning, and then the introduction and operation of the vector system was examined through fluorescence microscopy.

Specifically, donor cells were obtained from the kidney tissue of a KSP mini-pig (2-day-old, male), and the kidney tissue was cold-stored in DPBS wash buffer containing 10% (v/v) penicillin/streptomycin (Invitrogen) until isolation. Then, the kidney tissue was minced using a sterile surgical blade, placed on a 100-mm culture dish pretreated with gelatin (Sigma-Aldrich), and then cultured in Dulbecco's Modified Eagle's Medium (DMEM) (Invitrogen) containing 10% fetal bovine serum (FBS) (16000-044, GIBCO, Carlsbad, CA, USA), 10 ng/ml basic fibroblast growth factor (bFGF) and 1% penicillin/streptomycin until a sufficient amount of donor cells were obtained.

1 μg Cas9, 1 μg sgRNA and 2 μg RGS reporter plasmid DNA were premixed with 2×10⁶ donor cells in 100 μl of a buffer of Amaxa™ P3 primary cell 4D Nucleofector™ X Kit L (Lonza), and then the vector system was introduced into the cells by electroporation using the program EH-113 of 4D Nucleofector™ (Lonza). After 24 hours, cells expressing fluorescence were examined under a fluorescence microscope (Leica).

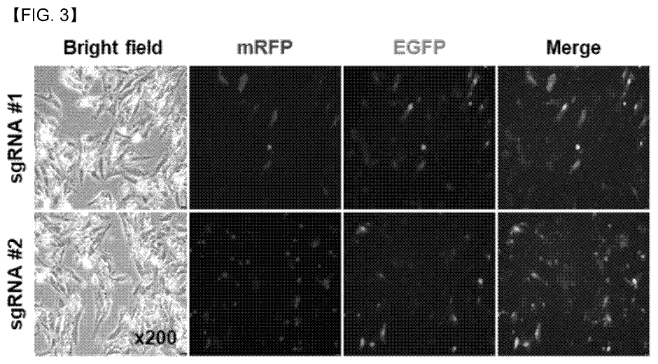

As a result, both red fluorescence and green fluorescence were detected in the donor cell line into which the JAK3 knock-out gene scissors expression vector system constructed in Experimental Example 1-2-1 was introduced. Thus, it was confirmed that the vector system of the present disclosure could be introduced into the donor cell line and could operate in the donor cell line (FIG. 3).

### Experimental Example 1-2-3. Verification of JAK3 Knock-out Gene Scissors Expression Vector System-Flow Cytometry

To verify the JAK3 knock-out gene scissors expression vector system, the JAK3 knock-out gene scissors expression vector system constructed in Experimental Example 1-2-1 was introduced into a normal mini-pig donor cell line for somatic cell cloning. Then, to confirm the introduction and operation of the vector system, flow cytometry was performed, and cells expressing red fluorescence and green fluorescence among all the cells were quantitatively analyzed.

Specifically, for flow cytometry, donor cells were transfected with red fluorescence, green fluorescence, and empty vectors to make controls, and then a wavelength band where red fluorescence and green fluorescence overlap was removed through a compensation process, and a setting value at which each intrinsic fluorescence is measured was obtained. Subsequently, under the set conditions, fluorescence expression was analyzed using donor cells into which the gene scissors vector system was introduced.

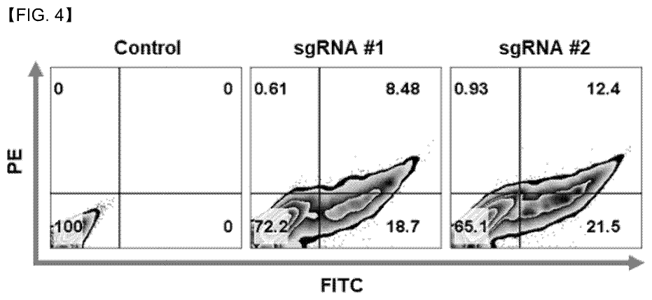

As a result, the activity of gene scissors was confirmed in the donor cell line into which the JAK3 knock-out gene scissors expression vector system constructed in Experimental Example 1-2-1 was introduced, compared to the non-transgenic donor cell line (sgRNA #1 and sgRNA #2 for each gRNA). Thus, it was confirmed that the vector system of the present disclosure could be introduced into the donor cell line and could operate in the donor cell line (FIG. 4).

### Experimental Example 1-2-4. Verification of JAK3 Knock-out Gene Scissors Expression Vector System-T7E1 Assay

To confirm whether the JAK3 knock-out gene scissors expression system is verified, a T7 endonuclease assay (T7E1 assay) was performed on a mini-pig donor cell line into which the JAK3 knock-out gene scissors expression vector system constructed in Experimental Example 1-2-3 was introduced, to determine whether selected mutations were generated.

Specifically, genomic DNA was isolated from mutant candidates (sgRNA #1 and sgRNA #2 for each gRNA) and a control cell line, and then predicted mutation sites were amplified by PCR. A control cell line-derived product and each of the mutant-derived products, from the amplified PCR product were mixed, and then the temperature was lowered by 2° C./s from 95° C. to 25° C. to form a type 2 heteroduplex shape, and the formed type 2 heteroduplex was treated with 2 units of T7E1, followed by electrophoresis on a 2% agarose gel, to confirm this.

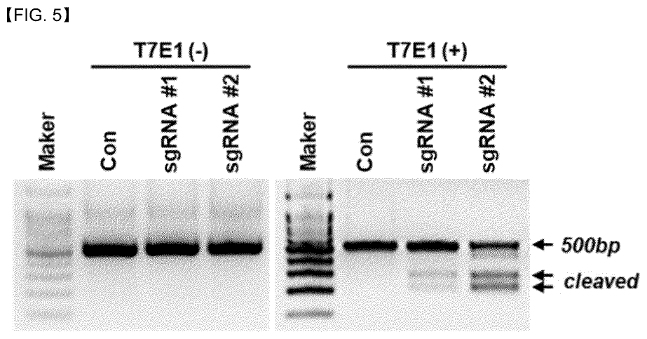

As a result, the cleavage of a DNA mixture product was not confirmed in all of the T7E1-untreated control, sgRNA #1, and sgRNA #2, whereas the cleavage of a DNA mixture product was confirmed in sgRNA #1 and sgRNA #2 that were treated with T7E1 (FIG. 5).

Through the above results, it was confirmed that sgRNA #2 can knock out the gene with higher efficiency, and sgRNA #2 was used in the following experiments.

### Experimental Example 1-3. Preparation of JAK3 Knock-out Transgenic Cell Line and Confirmation of Gene Knock-Out

The JAK3 knock-out gene scissors expression vector system was introduced into a normal mini-pig donor cell line for somatic cell cloning, and then the transgenic mini-pig donor cell line was cultured as single cells and subjected to sequencing analysis to confirm whether a mutation occurs in the gRNA-hybridizing JAK3 nucleic sequence by the JAK3 knock-out gene scissors expression vector system.

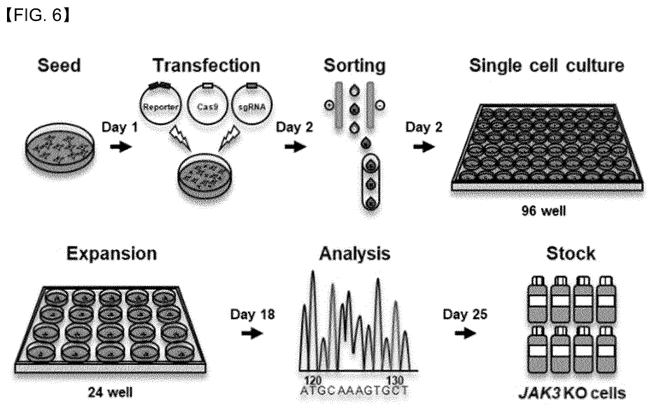

Specifically, cells expressing green fluorescence were sorted as single cells into a 96-well plate containing growth medium using a FACSAria III sorter (BD Biosciences). Two weeks after sorting, single colony cells selected from the 96-well plate were subcultured in a 6-well plate, and then genomic DNA was extracted from a portion of each cloned cell line using a DNeasy Blood & Tissue Kit (Qiagen). Primers were designed so that the size of a PCR product around the sgRNA of the JAK3 gene was about 500 bp, and the gene was amplified using PCR equipment (Applied Biosystems). The amplified PCR product was electrophoresed and then recovered by gel elution. To examine the form of indel (nucleotide sequence changes such as mutation and insertion), the nucleotide sequence of the DNA recovered by gel elution was analyzed using the primers (Table 1) used for PCR amplification (FIG. 6).

TABLE 1

| Primer | | |
|---|---|---|
| (Product size 500 bp) | | Primer sequence (5′→3′) |
| JAK3 | Forward | GCAGTATCGCTGCTCCCTAC |
| | Reverse | GAGAGCATCCCTTTCCACTG |

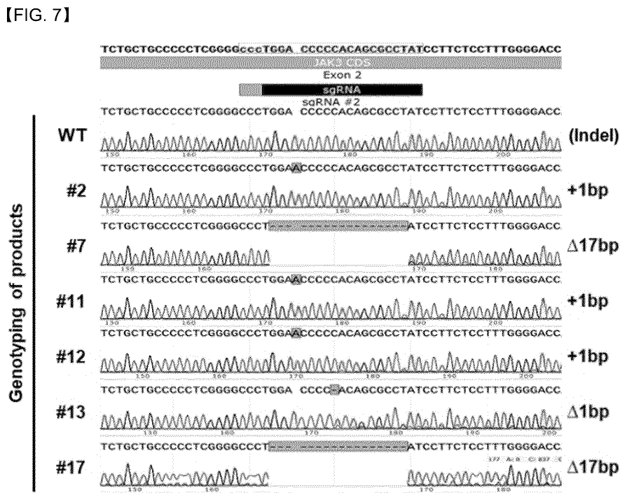

As a result, as illustrated in Table 2 and FIG. 7, it was confirmed that a mutation occurred in a nucleotide sequence corresponding to the sgRNA-hybridizing JAK3 nucleotide sequence in #2, #7, #11, #12, #13, and #17 among the donor cell lines.

TABLE 2

| Classification | Nucleotide sequence (5′→3′) | SEQ ID NO |
|---|---|---|
| gRNA-hybridizing | C CCTCGGGGCC CTGGA CCCCC ACAGCGCCTA T | |
| JAK3 sequence | | |
| #2 cell line JAK3 | TCTGCTGCCC CCTCGGGGCC CTGGAACCCCC | SEQ ID NO: |
| sequence (+1 bp) | ACAGCGCCTA TCCTTCTCCT TTGGGGACC | 4 |
| #7 cell line JAK3 | TCTGCTGCCC CCTCGGGGCC CTA TCCTTCTCCT | SEQ ID NO: |
| sequence (−17 bp) | TTGGGGACC | 6 |
| #11 cell line JAK3 | TCTGCTGCCC CCTCGGGGCC CTGGAACCCCC | SEQ ID NO: |
| sequence (+1 bp) | ACAGCGCCTA TCCTTCTCCT TTGGGGACC | 4 |
| #12 cell line JAK3 | TCTGCTGCCC CCTCGGGGCC CTGGAACCCCC | SEQ ID NO: |
| sequence (+1 bp) | ACAGCGCCTA TCCTTCTCCT TTGGGGACC | 4 |
| #13 cell line JAK3 | TCTGCTGCCC CCTCGGGGCC CTGGA CCCC ACAGCGCCTA | SEQ ID NO: |
| sequence (−1 bp) | TCCTTCTCCT TTGGGGACC | 5 |
| #17 cell line JAK3 | TCTGCTGCCC CCTCGGGGCC CTA TCCTTCTCCT | SEQ ID NO: |
| sequence (−17 bp) | TTGGGGACC | 6 |

### Experimental Example 1-4. Verification of JAK3 Knock-out Transgenic Cell Line-Amino Acid Sequencing Analysis

To confirm how the nucleotide sequence mutation in the gRNA-hybridizing JAK nucleotide sequence, identified in Experimental Example 1-3, affects the structure of a JAK3 protein, an amino acid encoded by the mutant JAK3 coding sequence in the transgenic donor cell line was analyzed.

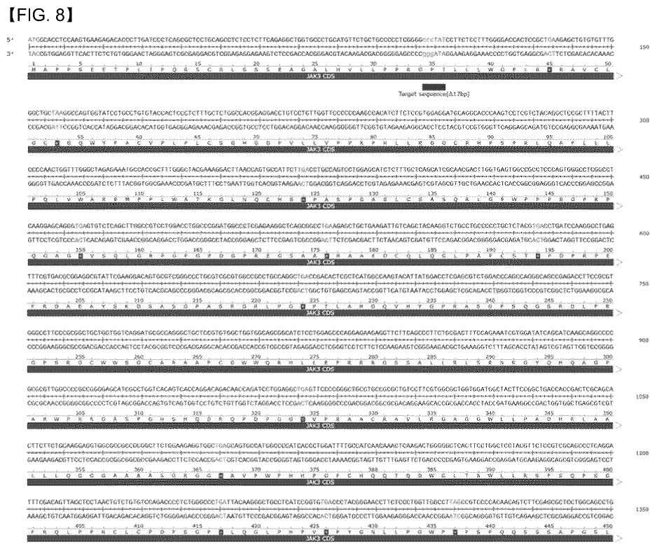

As a result, it was confirmed that, in the transgenic donor cell line #7 (−17 bp) (FIG. 8) in which the gRNA-hybridizing JAK3 nucleotide sequence was mutated, the amino acid codon encoding JAK3 was changed by nucleotide sequence deletion, and a premature stop codon was created. The stop codon is marked in red in FIG. 8.

### Experimental Example 1-5. Verification of JAK3 Knock-out Transgenic Cell Line-Karyotyping

To confirm whether the nucleotide sequence mutation in the gRNA-hybridizing JAK3 nucleotide sequence, identified in Experimental Example 1-3, affects the autosomal and sex chromosomes of the transgenic mini-pig donor cell line, the chromosomal karyotype of the transgenic donor cell line was analyzed through the G-banding karyotyping method and chromosomal stability was examined.

Specifically, when the cells reached a confluency of 70% to 80%, the cells were treated with 0.1 mg/ml colcemid (Sigma-Aldrich) in a 5% CO₂ incubator at 37° C. for 50 minutes to stop cell division in metaphase. The cells whose division was stopped were expanded by treatment with a low osmotic solution of 0.06 M KCl (Sigma-Aldrich) at 20° C. for 10 minutes, and fixed in a fixative of methanol/acetic acid (3:1) for 30 minutes, and then centrifuged at 150 g for 10 minutes. After the fixing procedure was repeated 3 times, the fixed cells were dropped onto a glass slide and air-dried overnight at 65° C. Finally, the cells were pretreated with 0.25% trypsin, and then the chromosomes were stained by Giemsa staining and karyotyped.

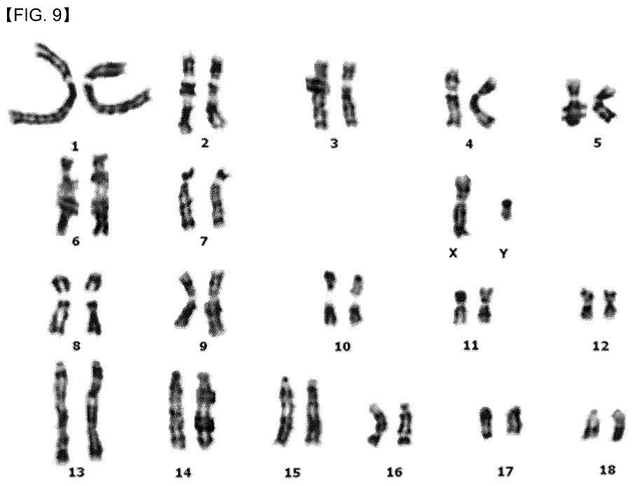

As a result, it was confirmed that the autosomal and sex chromosomes of transgenic donor cell line #7 (FIG. 9) in which the sgRNA-hybridizing JAK3 nucleotide sequence was mutated showed a normal chromosomal pattern, indicating that the transgenic mini-pig donor cell line retained a normal diploid karyotype.

### Experimental Example 1-6. Verification of JAK3 Knock-out Transgenic Cell Line-Off-Target Analysis

Off-target analysis was performed to confirm whether sgRNA #2 of the present disclosure acts on a nucleotide sequence other than JAK3.

Specifically, nucleotide sequencing and deep sequencing of genes including sequences similar to the JAK3 target sequence (a sequence complementary to the sgRNA-hybridizing JAK3 nucleotide sequence) were performed using the primers shown in Table 3, except that cells derived from the JAK3 knock-out transgenic cloned mini-pigs crushed to death were used.

TABLE 3

| Gene Name | | Primer sequence (5′→3′) |
|---|---|---|
| JAK3 On-target | | F: AGCCTGCAGTATCGCTGCTCC |
| (1^{st} PCR) | | R: GCTCTCAATCTGGGTTCCACATA |
| JAK3 On-target | | F: ACACTCTTTCCCTACACGACGCTCTTCCGATCTCAACTGAGGTGCTGTCT |
| (Adaptor, Nest | | CTTTCTGTGTCC |
| PCR) | | R: GTGACTGGAGTTCAGACGTGTGCTCTTCCGATCTCCTTCTAGAGACCCA |
| | | CTCACCACTGG |
| JAK3 | 7SK | F: CGGGCTTTCTGTTTTCTGAC |
| off- | | R: ATTCTGGAGGCAAGACAAGC |
| target | NA1 | F: ACGTAAATGGGAAGACTGC |
| (1^{st} | | R: AAAGGGCTTTGGTGGAATCT |
| PCR) | NA2 | F: TCCTGTGCGGTTGGATTTTCAG |
| | | R: TCCTCCTAGCTTCGACCTCA |
| | NA3 | F: TGGGGATCAGTTTCCTTTTG |
| | | R: AGAGGTTTTCAGACTCTAGAGCCCA |
| | NDUFA12 | F: CTTACATCTTCACATTTCAGAGCCC |
| | | R: ACCTTTTCTGTGAGGGATGC |
| | SARDH | F: CTCAGAGGTGGAGCAGGTGT |
| | | R: ACGTGCCCAGAGACCAGCTGTCC |
| | TMEM9 | F: TCTGAAACAGGCATCTGTGC |
| | | R: TGCTGGCCATGAGCCTAAACCAAC |
| | Unknown | F: TCCTCTCCCATTTTACTCCCTTCATT |
| | 1 | R: TTGCTGGGAGCTAAGGAGAA |
| | Unknown | F: CACAAGTGCTCAAAGGCAAA |
| | 2 | R: TCTTGCTGCTTTCAAGATTGTTCC |
| | Unknown | F: TTGGGAGCTGAGTTTTGAGC |
| | 3 | R: GCTCAAGGGCCTGCTAATGG |
| JAK3 | 7SK | F: ACACTCTTTCCCTACACGACGCTCTTCCGATCTCGGGCTTTCTGTT |
| off- | | TTCTGACATCGGTCA |
| target | | R: GTGACTGGAGTTCAGACGTGTGCTCTTCCGATCTATTCTGGAGGC |
| adaptor | | AAGACAAGCAAGCAG |
| (2^{nd} | NA1 | F: ACACTCTTTCCCTACACGACGCTCTTCCGATCTCTCACAGTTCAG |
| PCR) | | TTTTGGACCTGTTAACTAA |
| | | R: GTGACTGGAGTTCAGACGTGTGCTCTTCCGATCTGTGATGTTCAT |
| | | TAAGACAATTACCCAGCTTAAATTTGC |
| | NA2 | F: ACACTCTTTCCCTACACGACGCTCTTCCGATCTGTGCTCAGTGATGGAGTTT |
| | | GCTTATCTATTTA |
| | | R: GTGACTGGAGTTCAGACGTGTGCTCTTCCGATCTGATATGGGTCC |
| | | AAACCATTTTACAATCTTG |
| | NA3 | F: ACACTCTTTCCCTACACGACGCTCTTCCGATCTTGACCAGGCCCGACTGAAA |
| | | TAAAGCCC |
| | | R: GTGACTGGAGTTCAGACGTGTGCTCTTCCGATCTCAGGCCCTAGA |
| | | TCTCCATGAGACCTTCAAAAG |
| | NDUFA12 | F: ACACTCTTTCCCTACACGACGCTCTTCCGATCTCCATTCCTGACATCTTCTTC |
| | | CTGTGCTC |
| | | TGA |
| | | R:GTGACTGGAGTTCAGACGTGTGCTCTTCCGATCTAGGGAACATGGAA |
| | | CCGCTCTGGCATCTCCA |
| | SARDH | F: ACACTCTTTCCCTACACGACGCTCTTCCGATCTCCTTGGCCTCTGACTGCAG |
| | | ACATCTGACTGT |
| | | R: GTGACTGGAGTTCAGACGTGTGCTCTTCCGATCTCTTTCCTGCAT |
| | | GATGTTCTGGAGGGTTTTG |
| | TMEM9 | F: ACACTCTTTCCCTACACGACGCTCTTCCGATCTGCAGGAAGACAAGGGTCT |
| | | GCCTGCGGG |
| | | R: GTGACTGGAGTTCAGACGTGTGCTCTTCCGATCTCCTGCCCAGC |
| | | CCTGGAGCTGTGTC |
| | Unknown | F: ACACTCTTTCCCTACACGACGCTCTTCCGATCTGCACAGAATCCAAACCCCT |
| | 1 | ACAGCTACTCA |
| | | R: GTGACTGGAGTTCAGACGTGTGCTCTTCCGATCTAGGAGAAGTAA |
| | | TGACAAATTCACGTGAGG |
| | Unknown | F: ACACTCTTTCCCTACACGACGCTCTTCCGATCTCTGCTGCTCGCTGGCCTTG |
| | 2 | GCC |
| | | R: GTGACTGGAGTTCAGACGTGTGCTCTTCCGATCTTCCTCCTTGGA |
| | | GTTCATTGAACTTTCTGAAGA |
| | Unknown | F: ACACTCTTTCCCTACACGACGCTCTTCCGATCTAGCAGCAGAAGAGCAGGA |
| | 3 | GGTCTCTGTGG |
| | | R: GTGACTGGAGTTCAGACGTGTGCTCTTCCGATCTTTTTAATCCTGGACAGAG |
| | | CAGTAATGGCGT |
| RAG2 On-target | | F: TTCTTTACCCAGCTGCCTGGATTT |
| (1^{st} PCR) | | R: CTTCAGTTTGAGATGGTTATGCTTT |
| RAG2 On-target | | F: ACACTCTTTCCCTACACGACGCTCTTCCGATCTTTTTAACAGGCTT |
| (Adaptor, Nest | | TTTATGTGTGAGGGATCTAAACA |
| PCR) | | R: GTGACTGGAGTTCAGACGTGTGCTCTTCCGATCTGGCCAGCCTTT |
| | | TTGGCC AAAGAAGAAGA |
| RAG2 | FGF14 | F: CCACCACGAATAGAGGGATG |
| off- | | R: CCCCACTTTGGATGCTCTTA |
| target | MACF1 | F: TATACCCGGAAGTGGGATTG |
| (1^{st} | | R: GCCATCGGGTATTATGGAAA |
| PCR) | NA | F: CGGAAAGAAAGGTAAACCCTCGAG |
| | | R: AAATATTTTAAGAGTTCCCTGGTGGCT |
| | SRBD1 | F: TTGTGGTGGTATGAACTGAACC |
| | | R: TCCCTTCCACAGAAGGGTATTATT |
| | U6 | F: GCAAAATATTGGGAAACATGG |
| | | R: GCAACCTACACCACAGCTCA |
| RAG2 | FGF14 | F: ACACTCTTTCCCTACACGACGCTCTTCCGATCTCCACCACGAATA |
| off- | | GAGGGATGTAAACCTTCGTT |
| target | | R: GTGACTGGAGTTCAGACGTGTGCTCTTCCGATCTCTTTAAATAGC |
| adaptor | | TGCACA GTATGGGTTCTTTCCG |
| (2^{nd} | MACF1 | F: ACACTCTTTCCCTACACGACGCTCTTCCGATCTCCGGAAGTGGGA |
| PCR) | | TTGCTAGATCCTGTGGTA |
| | | R: GTGACTGGAGTTCAGACGTGTGCTCTTCCGATCTCATCGGGTATT |
| | | ATGGAAAGGTGTTCAACATCACTA |
| | NA | F: ACACTCTTTCCCTACACGACGCTCTTCCGATCTTCCATTCATGTGAATTTCAA |
| | | GAACAGGCAA AACTG |
| | | R: GTGACTGGAGTTCAGACGTGTGCTCTTCCGATCTGGCTCTAGTTA |
| | | CGGCTGTGGTGCGGGTTG |
| | SRBD1 | F: ACACTCTTTCCCTACACGACGCTCTTCCGATCTACCCTCAGTATCTATGAGG |
| | | CATGCCTA |
| | | R: GTGACTGGAGTTCAGACGTGTGCTCTTCCGATCTTCCCTTCCACA |
| | | GAAGGGTATTATTTGAGTAGAATAT |
| | U6 | F: ACACTCTTTCCCTACACGACGCTCTTCCGATCTGCAAAATATTGGGAAACAT |
| | | GGATATAAGGC |
| | | R: GTGACTGGAGTTCAGACGTGTGCTCTTCCGATCTGCAACGCCGG |
| | | ATCGTTAACCCACTGAGCA A |

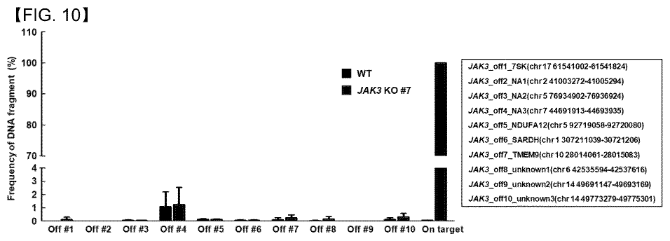

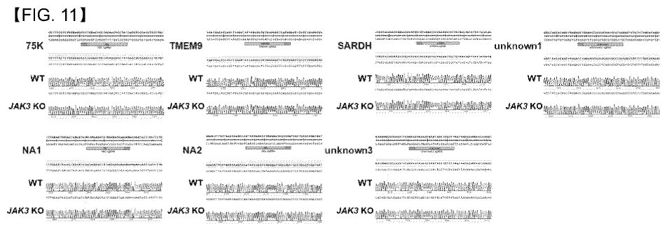

As a result, as confirmed in Table 4 and FIGS. 10 and 11, it can be seen that sgRNA #2 did not act in parts other than the JAK3 sequence, indicating that the sgRNA of the present disclosure acts specifically for JAK3.

TABLE 4

| | | | | | | | gene | |
|---|---|---|---|---|---|---|---|---|
| Coordinates | strand | MM | target_seq | PAM | distance | | name | gene id |
| chr2:26716430- | + | 0 | GATAACAG[TTGGTAATAACA] | TGG | 0 | E | RAG2 | ENSSSCG00000013287 |
| 26716452 | | | | | | | | |
| chr2:97990072- | + | 3 | AATAACAG[ATGATAATAACA] | TGG | 81312 | | U6 | ENSSSCG00000026711 |
| 97990094 | | | | | | | | |
| chr8:80554206- | + | 3 | GGTGACAG[TTGGTATTAACA] | GGG | NA | | NA | NA |
| 80554228 | | | | | | | | |
| Chr3:101066767- | + | 3 | GATGACAG[TAGGGAATAACA} | AGG | 1578 | I | SRBD1 | ENSSSCG00000008445 |
| 101066789 | | | | | | | | |
| chr11:77682210- | + | 3 | GATAAAAG[TTGATAAAAACA] | AGG | 4986 | I | FGF14 | ENSSSCG00000009527 |
| 77682232 | | | | | | | | |
| chr6:77682210- | − | 3 | GATAAGAG[TTCGTGAAAACA] | TGG | 1174 | I | IMACF1 | ENSSSCG00000003654 |
| 88224408 | | | | | | | | |

### Example 1: Production of JAK3 Knock-out Transgenic Cloned Mini-Pigs

Among the JAK3 knock-out transgenic donor cell lines constructed in Experimental Example 1-3, donor cell line #7, confirmed to have a better embryo development rate when somatic cell nuclear transfer (SCNT) was performed using embryos, was used to produce JAK3 knock-out transgenic cloned mini-pigs.

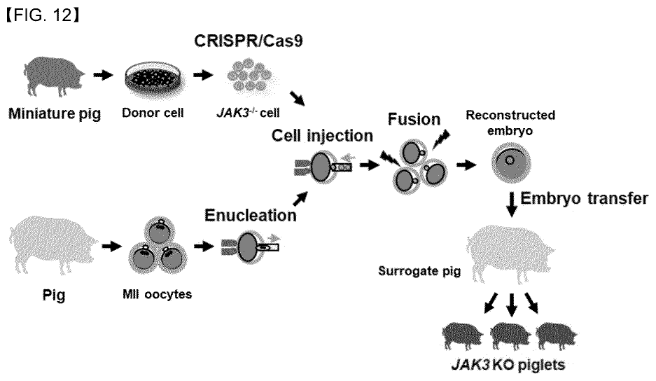

To this end, the method confirmed in FIG. 12 was performed.

First, a somatic cell nucleus of JAK3 knock-out transgenic donor cell line #7 was transferred into an enucleated egg.

Specifically, after in vitro maturation (IVM), matured oocytes with a visible first polar body were selected for SCNT. Cumulus cells-removed oocytes were placed in a pipette in DPBS containing 4 mg/ml BSA, 75 μg/ml penicillin G, 50 μg/ml streptomycin sulfate, and 7.5 μg/ml CB, the pipette is cut to form a slit, and the cytoplasm and the first polar body were removed from the ooctyes under an automated phase-contrast microscope (DMI 6000B, Leica) equipped with about 10% (NT-88-V3, Nikon-Narishige). The donor cells were cultured for 3 days without changing the medium so as to be synchronized in the G0/G1 phage of the cell cycle, and the oocytes were maintained in IVC medium in an incubator with 5% CO₂ at 38.5° C. until electrical fusion. The single donor cell/oocyte was equilibrated in a fusion medium consisting of 280 mM mannitol (Sigma-Aldrich) containing 0.1 mM MgSO₄·7H₂O and 0.01% PVA, and placed between two attached parallel electrodes (100 μm in diameter, CUY 5100-100, Nepagene). The oocytes were activated by a single direct current pulse of 23 V for 50 ρsec using an electro cell fusion generator, and then placed in an incubator with 5% CO₂ at 38.5° C. After 2 hours, the completely fused oocyte pair were selected under a phase-contrast microscope, transferred to a 1-mm gap wire chamber overlaid with 10 μl of 280 mM mannitol solution containing 0.1 mM MgSO₄·7H₂O, 0.1 mM CaCl₂·2H₂O, 0.5 mM HEPES, and 0.01% PVA, and then activated with 110 V DC for 50 ρsec using an electro cell fusion generator. To chemically activate the oocytes activated by electrical fusion, the oocytes were added to a medium containing 50 nM trichostatin A (TSA) and maintained in an incubator with 5% CO₂ at 38.5° C. for 4 hours. After 4 hours, the activated embryos were transferred to an IVC medium supplemented with 50 nM TSA and further cultured for 20 hours. After 20 hours, the SCNT embryos were washed in IVC medium, and placed in an incubator with 5% CO₂ at 38.5° C. Thereafter, division and blastocyst formation were evaluated on day 2 and day 6, respectively, and transfer into a surrogate pig was prepared.

Thereafter, the oocytes into which the somatic cell nucleus of JAK3 knock-out transgenic donor cell line #7 was transferred were transferred into a surrogate pig.

Specifically, the fused cloned embryos were cultured for 1 to 2 days in a 4-well plate containing 500 μl of culture medium, and then placed in a freezing tube containing 2 ml of the same culture medium and transferred to an embryo transfer place by an embryo transfer kit (Minitube, USA) warmed to 39° C. Surrogate pigs for transfer of cloned embryos were prepared by selecting animals whose estrus started. The surrogate pigs were washed and temporarily anesthetized by administering 0.5 g of pentothal sodium (JW Pharmaceutical Co., Ltd.) to the ear vein. Each of the anesthetized surrogate pigs was fixed to an operating table, and then subjected to inhalation anesthesia with 5% isoflurane. The anesthetized surrogate pig was prepared by incising the abdominal wall along the midline by about 5 cm and then exposing the uterus and ovaries to the outside. At this time, the cloned embryos were transferred by aspirating the same into a catheter (Tom cat catheter, Monoject, USA) and pushing the same to the isthmus of the fallopian tube. A total of 600 cloned embryos were transferred into two surrogate pigs for the production of JAK3 knock-out transgenic cloned pigs. After completion of cloned embryo transfer, the surrogate pigs were subjected to a pregnancy test using ultrasound on day 30. The surrogate pigs confirmed to be pregnant were subjected to hysterectomy 1-2 days before the 114th day to obtain offspring.

The surrogate pigs were bred in SPF (Special Pathogen-Free) facilities while the movement of the mini-pigs was eased using a flooring material made of plastic in consideration of the characteristics of the mini-pigs. In addition, a temperature-keeping pad was provided on the bottom of the breeding cage to help maintain mini-piglets at a constant temperature. JAK3 knock-out transgenic cloned mini-pigs were subjected to artificial suckling with feed containing 70 g safe milk, 1 g colostrum stick and 1 g vitamin in 500 ml water at 2-hour intervals from 2 days after delivery, and were subjected to iron injection twice. After birth of the mini-pigs, the individual weight and feed amount of each mini-pig were recorded for 3 months, and systematic maintenance and control of the bred IGF-1 knock-out mini-pigs was performed through microbial monitoring.

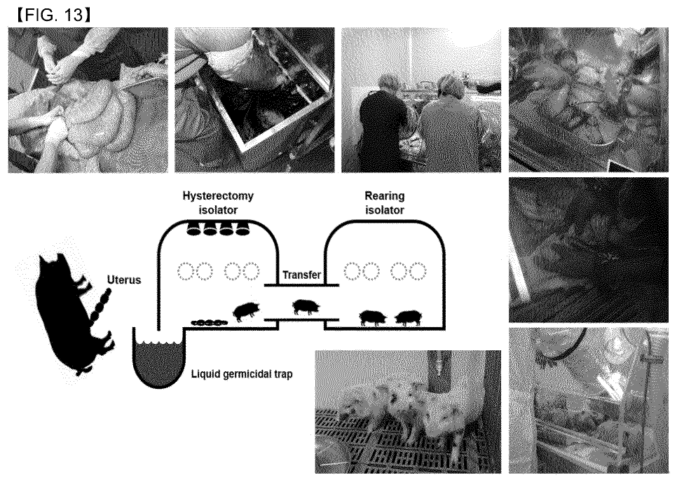

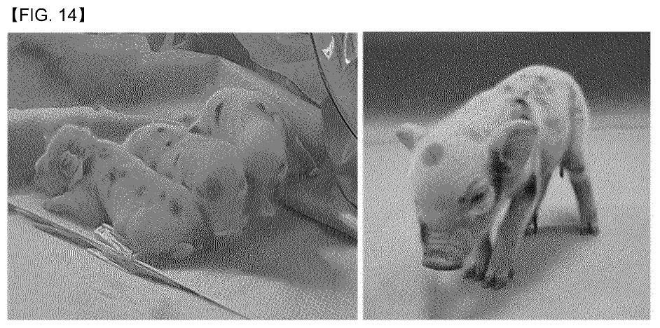

As a result, as confirmed in FIGS. 13 and 14, JAK3 knock-out min-pigs were produced.

### Experimental Example 2: JAK3 Gene Analysis of JAK3 Knock-out Transgenic Cloned Mini-Pigs

### Experimental Example 2-1. Confirmation of JAK3 Knock-out In JAK3 Knock-out Transgenic Cloned Mini-Pigs

To confirm whether the produced cloned mini-pigs are JAK3 knock-out transgenic mini-pigs, the nucleotide sequences of the JAK3 knock-out cloned mini-pigs were analyzed.

Specifically, nucleotide sequencing was performed in the same manner as in Example 2-1, except that cells derived from the JAK3 knock-out transgenic cloned mini-pigs produced in Example 1 and JAK3 knock-out transgenic donor cell line #7 were used.

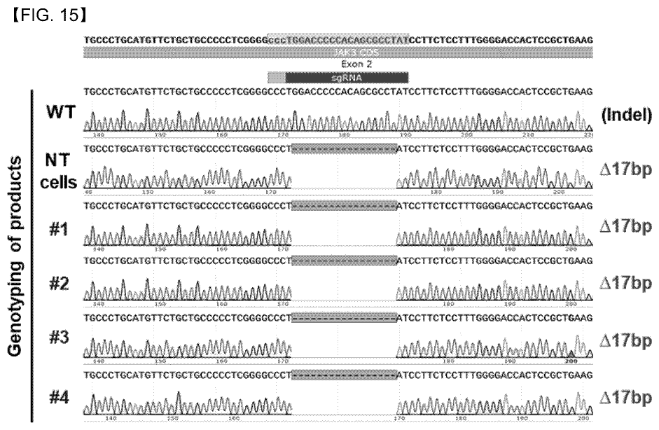

As a result, as can be confirmed in FIG. 15, it was confirmed that the gRNA-hybridizing JAK3 nucleotide sequences in all of the aborted, died and alive JAK3 knock-out transgenic cloned mini-pigs were the same as the nucleotide sequence of transgenic donor cell line #7 (−17 bp), indicating that the produced cloned mini-pigs were JAK3 knock-out transgenic cloned mini-pigs.

### Experimental Example 2-2. mRNA Analysis of JAK3 Knock-out Transgenic Cloned Min-Pigs

### Experimental Example 2-2-1. mRNA Analysis of JAK3 Knock-out Transgenic Cloned Mini-Pigs (RT-PCR)

To confirm the expression of JAK3 in the spleen or liver of the JAK3 knock-out transgenic cloned mini-pigs produced in Example 1, mRNA was extracted from the spleen and liver tissues of normal individuals and the JAK3 knock-out transgenic cloned mini-pigs produced in Example 1 and the expression thereof was analyzed.

Specifically, total RNA was extracted from cells and tissues using an RNeasy plus mini kit (QIAGEN) according to the method provided by the manufacturer. Total RNA (1 μg) was used for cDNA synthesis using a PrimeScript RT reagent kit (TAKARA), and the relative expression level of the gene was measured by real-time RT-PCR using a SYBR Premix Ex Taq II (TAKARA), and analyzed by a StepOnePlus Real-Time PCR System (Applied Biosystems). The reaction for quantitative RT-PCR analysis was performed under the following conditions: 95° C. for 10 min; 40 cycles of 95° C. for 30 sec, 62° C. for sec and 72° C. for 30 sec; and last 72° C. for 5 min. For comparative analysis, the mRNA expression level was normalized by GAPDH and then expressed as fold-change. WT was used as a control ΔCt (CΔCt) value, and a sample delta Ct (SΔCt) value was calculated from the difference between GAPDH and the Ct value of the target gene. The relative gene expression level between the sample and the control was determined using the equation 2-(SΔCt-CΔCt).

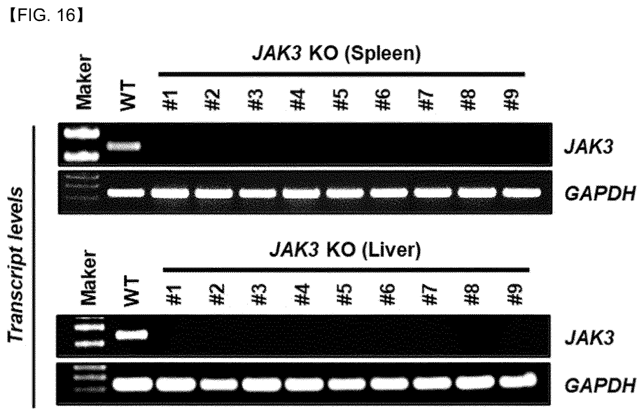

As a result, JAK3 expression was not observed in the JAK3 knock-out transgenic cloned mini-pigs (FIG. 16).

### Experimental Example 2-2-2. mRNA Analysis of JAK3 Knock-out Transgenic Cloned Mini-Pigs (Western Blot)

To confirm the expression of JAK3 in the spleen or liver of the JAK3 knock-out transgenic cloned mini-pigs produced in Example 1, mRNA was extracted from the spleen tissues of normal individuals and the JAK3 knock-out transgenic cloned mini-pigs produced in Example 1 and the expression thereof was analyzed.

For Western blot analysis, 30-60 μg of protein lysates were separated on 8-12% sodium dodecyl sulfate-polyacrylamide gels and transferred to 0.45 μm nitrocellulose membranes (Cat #HAWP04700, EMD Millipore, Burlington, MA, USA). The membranes were blocked with 5% skim milk (Cat #232100, BD Biosciences) or bovine serum albumin (Cat #A9647, Sigma) in 10 mM Tris pH 7.4, 150 mM NaCl and 0.02% Tween-20 (TBST), and then incubated overnight at 4° C. with primary antibodies (JAK3, Cat #ARP54306_P050, Aviva Systems Biology, San Diego, CA, USA, 1:1000; pSTAT5, Cat #71-6900, Thermo Fisher Scientific, 1:1000; STATS, Cat #66459-1-Ig, Proteintech, Manchester, UK, 1:1000; and GAPDH, Cat #LF-MA0038, λB Frontier, Seoul, Republic of Korea, 1:2000). The membranes were washed three times with TBST, and then blocked with 5% skim milk in TBST, and incubated at room temperature for 1 hour using horseradish peroxidase-conjugated secondary antibodies. The blots were washed with TBST, and then antibody binding was examined using SuperSignal West Pico PLUS Chemiluminescent Substrate (Cat #34580, Thermo Fisher Scientific) according to the manufacturer's instructions. Results were obtained through at least three independent experiments.

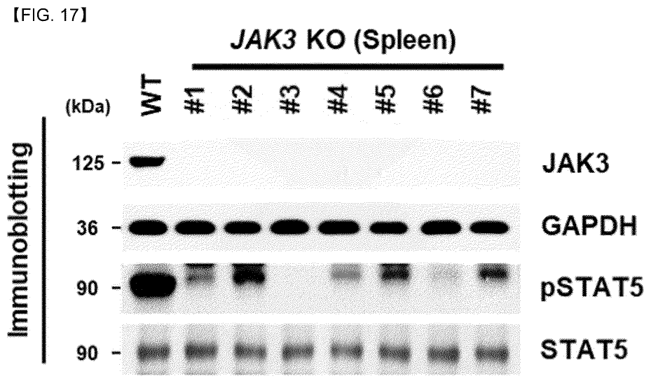

As a result, JAK3 expression was not observed in the JAK3 knock-out transgenic cloned mini-pigs (FIG. 17).

### Experimental Example 3: Phenotypic Analysis of JAK3 Knock-out Transgenic Cloned Mini-Pigs (1)

### Experimental Example 3-1. Identification of Thymus, Spleen, and Submandibular or Mesenteric Lymph Nodes

The thymus, spleen, and submandibular or mesenteric lymph nodes of the JAK3 knock-out transgenic cloned mini-pigs produced in Example 1 were examined.

Specifically, normal individuals in which JAK3 was not knocked out (wild type; WT), RAG2 knock-out transgenic cloned mini-pigs (RAG2 knock out; RAG2 KO), and the JAK3 knock-out transgenic cloned mini-pigs of Example 1 (JAK3 knock-out; JAK3 KO) were dissected and the thymus, spleen, and submandibular or mesenteric lymph nodes were visually confirmed.

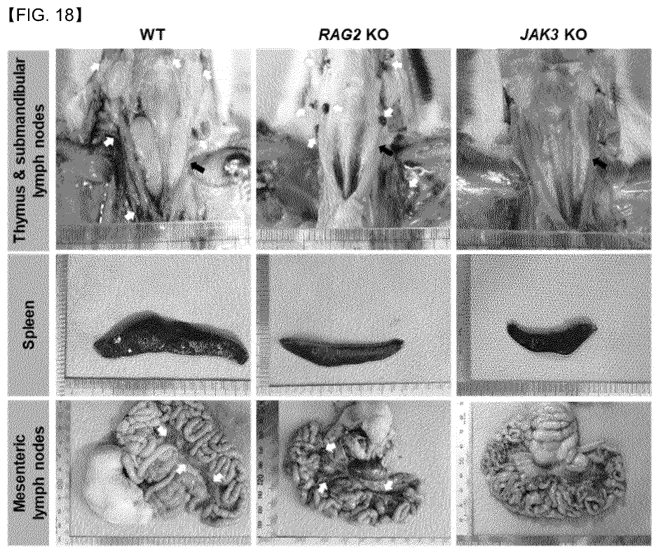

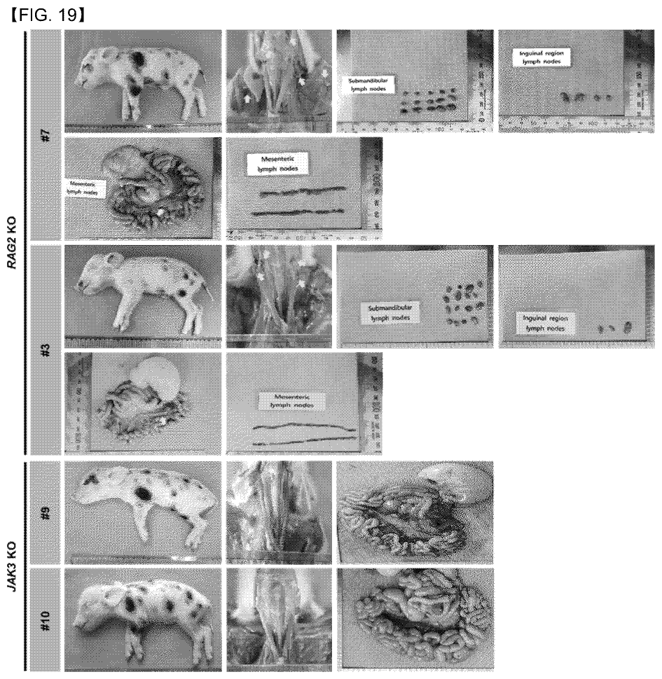

As a result, some thymuses (blue arrow) remained in the RAG2 KO mini-pigs, whereas no thymus was observed in the JAK3 KO mini-pigs, and the size and weight of the spleen were remarkably reduced in the JAK3 KO mini-pigs compared to the RAG2 KO mini-pigs. In addition, also in the lymph nodes, submandibular and mesenteric lymph nodes were observed in the RAG2 KO mini-pigs, although decreased, compared to normal individuals. However, no submandibular and mesenteric lymph nodes were observed in the JAK3 KO mini-pigs, from which it was confirmed that severe combined immunodeficiency was induced more severely (see FIGS. 18 and 19).

### Experimental Example 3-2. Confirmation of Tissue Formation in Thymus and Spleen (Immunohistochemical Staining)

The thymus and spleen of normal individuals in which JAK3 was not knocked out (wild type; WT), obtained in Experimental Example 3-1, the RAG2 knock-out transgenic mini-pigs (RAG2 knock out; RAG2 KO), and the JAK3 knock-out transgenic cloned mini-pigs of Example 1 were immunohistochemically stained to determine whether tissue structures related to immune cells were formed.

For immunohistochemical staining, the tissues were fixed with 10% (w/v) neutral buffered formalin (Cat #0151, BBC Biochemical, Mount Vernon, WA, USA) at a pH of 6.8-7.2. Then, the tissues were embedded in paraffin, and tissue sections were cut to a thickness of 3-5 μm on glass slides. Peroxidase activity was first blocked by treating the tissue sections with 1% hydrogen peroxide for 10 minutes. Samples were then pretreated with citrate buffer, pH 6.0 (Cat #ab94674, Abcam Inc., Cambridge, MA, USA) or proteinase K for antigen retrieval, and blocked in BSA solution (Sigma-Aldrich). Thereafter, the samples are washed and incubated overnight with primary antibodies (shown in Supplementary Table 11). After incubation, each sample was washed again, and then incubated together with horseradish peroxidase-conjugated secondary antibodies. A VECTASTAIN Elite λBC kit (Vector Laboratories, Burlingame, CA, USA) was used for detection and staining signals were visualized using 3,3-diaminobenzidine (Cat #54-10-00, KPL, Gaithersburg, MD, USA). Background staining was performed with hematoxylin. All microscope images were acquired using Leica DM2500 (Leica Microsystems).

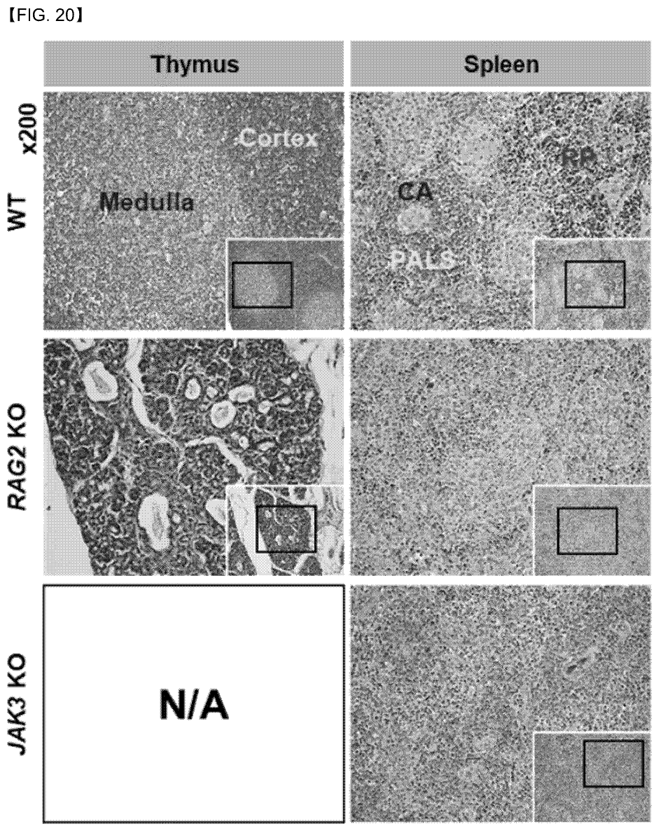

As a result of histological analysis of the thymus and the spleen, it was confirmed that neither the RAG2 KO mini-pigs nor the JAK3 KO mini-pigs did not form a tissue structure in which normal immune cells can be formed (FIG. 20).

### Experimental Example 3-3. Immunohistochemical Staining and Flow Cytometry of Spleen Tissue

B, T, and NK cells were identified through flow cytometry in the blood (peripheral blood mononuclear cell; PBMC) obtained by immunohistochemistry and removal of red blood cells from the spleen tissue obtained in Experimental Example 3-2.

Specifically, immunohistochemical staining was performed in the same manner as in Experimental Example 3-2 except that the antibodies shown in Table 5 were used, and flow cytometry was performed using a method similar to that in 1-2-3 as described above as follows, except that the antibodies shown in Table 6 were used.

TABLE 5

| | Target | | | | |
|---|---|---|---|---|---|
| Antigen | population | Clone | Isotype | Dilution | Suppler |
| CD3 | Pan-T-cells | SP7 | Rabbit IgG | 1:200 | Abcam |
| CD20 | B lymphocytes | EP459Y | Rabbit IgG | 1:250 | Abcam |
| CD335 | NKp46 | VIV-KM1 | Mouse IgG1 | 1:200 | Invitrogen |
| IgA | Plasma cells | | Goat IgA | 1:50 | BETHYL |

For flow cytometry, the spleen sections of euthanized WT, RAG2 KO pigs, and JAK3 KO pigs were collected in RPMI 1640 medium supplemented with 10% FBS for identification of T and B lymphocytes. It was then minced with a soft MACS Dissociator (Cat #130-093-235, Miltenyi Biotec, Bergisch Gladbach, Germany) and aspirated several times through a 20-gauge needle and allowed to pass through a 70 um nylon mesh cell strainer. Subsequently, the spleen cell suspension was incubated with Pharm Lyse solution (BD Biosciences) for 15 minutes to lyse the red blood cells and then pelleted at 200×g for 5 minutes. After the supernatant was removed, the pellet was resuspended in DPBS containing 2% FBS (Gibco) and cells were counted in an automatic cell counter (Cat #R1 Olympus Corporation, Tokyo, Japan). Subsequently, the cells were resuspended and divided into aliquots of 1×10⁶ cells in 200 μl of staining buffer. FITC-conjugated mouse anti-porcine CD21, mouse anti-porcine CD3ε, and mouse anti-T−2 mycotoxin IgG1 κ (isotype control) (Southern Biotech, Birmingham, AL, USA) were added to the cells at 0.5 μg per 1×10⁶ cells. The cells were incubated in the dark at 4° C. for 30 minutes. The cells were then washed twice and resuspended in fresh staining buffer. The cells were analyzed by FACS (BD) and data was analyzed using FlowJo software (FlowJo, LLC, Ashland, OR, USA).

TABLE 6

| Antigen | Conjugate | Clone | Isotype | Dilution | Suppler |
|---|---|---|---|---|---|
| Isotype | PE/Cy7 | | Mouse BALB/c | 1:200 | BD |
| Control | | | IgG1, _{K} | | |
| Isotype | Alexa647 | | Mouse BALB/c | 1:200 | BD |
| Control | | | IgG2a, _{K} | | |
| Isotype | PE | | Mouse BALB/c | 1:200 | BD |
| Control | | | IgG2b, _{K} | | |
| CD3 | PE/Cy5 | PPT3 | Mouse IgG1 | 1:200 | Abcam |
| CD4a | PE/Cy7 | 74-12-04 | Mouse BALB/c | 1:200 | BD |
| | | | IgG2b, _{K} | | |
| CD8a | Alexa647 | 76-2-11 | Mouse BALB/c | 1:200 | BD |
| | | | IgG2a, _{K} | | |
| CD14 | FITC | MIL2 | Mouse IgG2b | 1:200 | Bio-Rad |
| CD16 | PE | G7 | Mouse IgG1 | 1:200 | Bio-Rad |
| CD45RA | FITC | MIL13 | Mouse IgG1 | 1:200 | AbDserotec |
| CD172a | PE | 74-22-15A | Mouse BALB/c | 1:200 | BD |
| | | | IgG2b, _{K} | | |

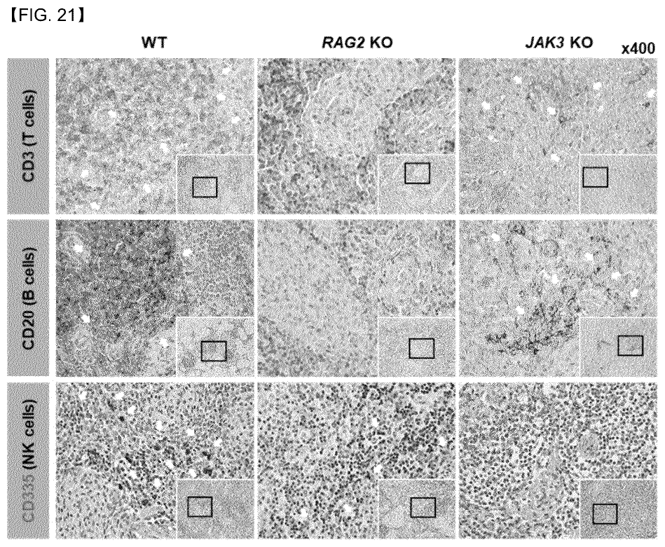

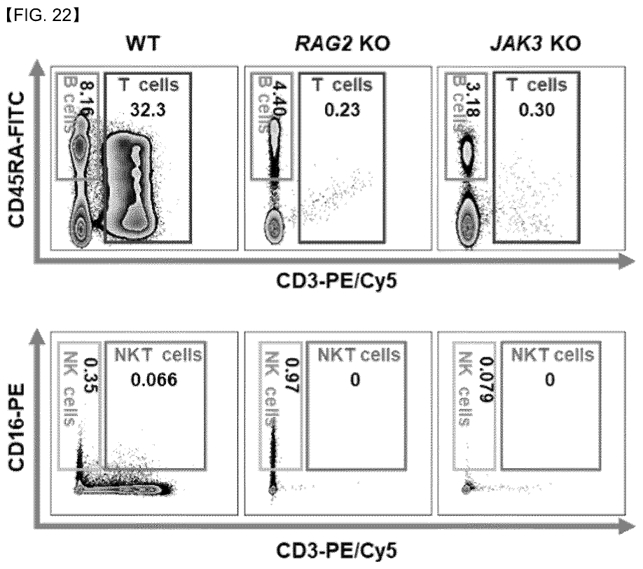

As a result, in the RAG2 KO mini-pigs, T and B cells were deficient, but NK cells were observed, whereas deficient NK cells and reduced T and B cells were confirmed in the JAK3 KO mini-pigs (yellow arrow+staining) (FIGS. 21 and 22).

### Experimental Example 3-4. Confirmation of Organ Weight

The body weight and the weights of the thymus and the spleen of the JAK3 knock-out transgenic cloned mini-pigs produced in Example 1 were examined.

Specifically, the body weight and the weights of the thymus and the spleen of normal individuals in which JAK3 was not knocked out (wild type; WT), RAG2 knock-out transgenic cloned mini-pigs (RAG2 knock out; RAG2 KO), and the JAK3 knock-out transgenic cloned mini-pigs of Example 1 (JAK3 knock-out; JAK3 KO) were measured.

As a result, as confirmed in Table 7, the body weight and the weight of the spleen of the JAK3 knock-out transgenic cloned mini-pigs were decreased compared to the normal individuals in which JAK3 was not knocked out (wild type; WT) and RAG2 knock-out transgenic cloned mini-pigs (RAG2 knock out; RAG2 KO), and it was confirmed that no thymus was formed in the JAK3 KO mini-pigs as described above.

TABLE 7
The weight of thymus and spleen organs of WT,
RAG2 KO, and JAK3 KO at autopsy

| Information | | Weight (g) | | | Sampling |
|---|---|---|---|---|---|
| Tg type | ID | Body | Thymus | Spleen | (day) |
| WT | #10 | 3100 | 8.410 | 12.290 | 19 |
| | #12 | 3700 | 11.967 | 9.372 | 19 |
| | #26 | 1593 | 2.945 | 5.170 | 5 |
| | #27 | 1112 | 1.978 | 2.354 | 4 |
| RAG2KO | #3 | 712 | 0.127 | 1.066 | 5 |
| | #5 | 850 | 0.083 | 1.039 | 6 |
| | #7 | 785 | 0.113 | 1.486 | 6 |
| | #8 | 658 | 0.180 | 1.065 | 4 |
| | #11 | 983 | 1.227 | 1.835 | 1 |
| JAK3 KO | #1 | 500 | 0 | 0.676 | 3 |
| | #2 | 562 | 0 | 0.752 | 3 |
| | #6 | 623 | 0 | 0.954 | 1 |
| | #7 | 586 | 0 | 0.802 | 3 |
| | #8 | 695 | 0 | 0.900 | 3 |
| | #9 | 450 | 0 | 0.745 | 3 |
| | #10 | 478 | 0 | 0.509 | 5 |
| | #12 | 692 | 0 | 0.678 | 5 |

### Experimental Example 3-5. Confirmation of Immune Cell Formation—V(D)J Recombination Detection Method

In the JAK3 knock-out cloned mini-pigs (JAK3 knock out; JAK3 KO) of Example 1 of the present disclosure, some T and B cells were formed in splenocytes (3-3. above), and it was confirmed whether these T and B cells can produce normal immune cells.

Specifically, for analysis of TCR and BCR V (D) J rearrangements, DNA samples were first extracted from the thymus, spleen and bone marrow of WT, RAG2 KO, and JAK3 KO pigs. Next, PCR was performed using the primers listed in Table 8. TCR recombination was analyzed by the following PCR conditions using Premixed Ex-Taq (Cat #RR005A, Takara Bio Inc): initial denaturation at 98° C. for 5 min; 30 cycles of 10 seconds at 98° C., 30 seconds at 68° C., and 1 minute at 68° C.; and the final elongation (synthesis) process at 68° C. for 7 minutes, to confirm the rearranged TCR band. In addition, to confirm the rearrangement of the BCR IgH gene, PCR was performed using Ex-Taq pre-mixed with FR1 and JH primers. PCR conditions were as follows: initial denaturation at 94° C. for 5 min; 30 cycles of 30 seconds at 98° C., 30 seconds at 64° C., and 30 seconds at 72° C.; the final extension at 72° C. for 7 minutes, to confirm the rearranged BCR band.

TABLE 8

| Gene Name | Accession No. | Primer sequence (5′→3′) | Product size |
|---|---|---|---|
| TCR-D1J1 | AB513625 | F: GCTGCTCTGGTGGTTTCTCAC | 1399 bp (germinal) |
| | | R: CACCAGTGCCCAAGTCTTAGC | 1162 bp (rearrangement 1) |
| | | | 788 bp (rearrangement 2) |
| | | | 701 bp (rearrangement 3) |
| | | | 464 bp (rearrangement 4) |
| BCR | AB079894.1 | F: CTGAGAACTCACGTCCAGTGC | 1691 bp (germinal) |
| | | R: CTGGCCCTAGACCTTTAGACC | |
| | | F: GAGGAGAAGCTGGTGGAGT | 436 bp (rearrangement) |
| | | R: TGAGGACACGACGACTTCAA | 442 bp (rearrangement) |

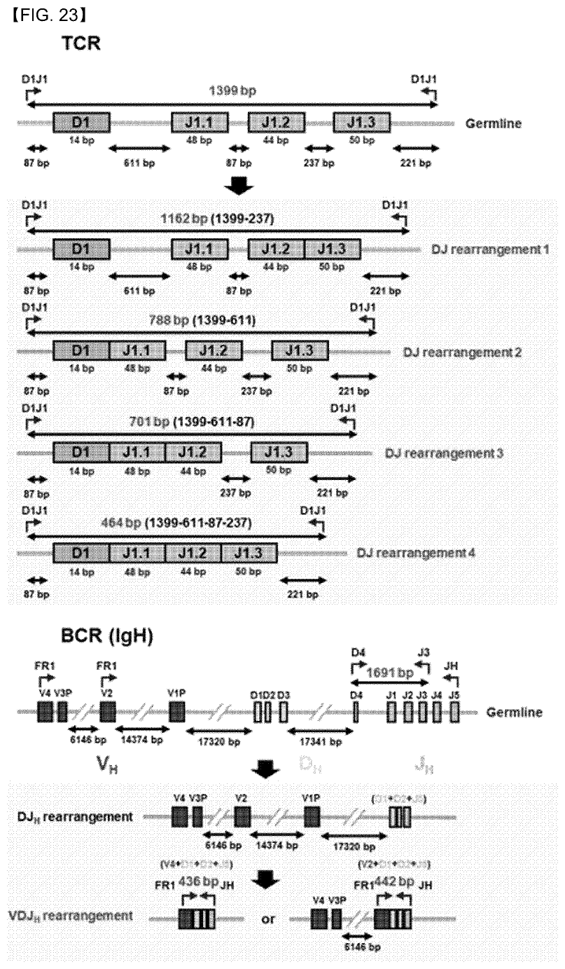

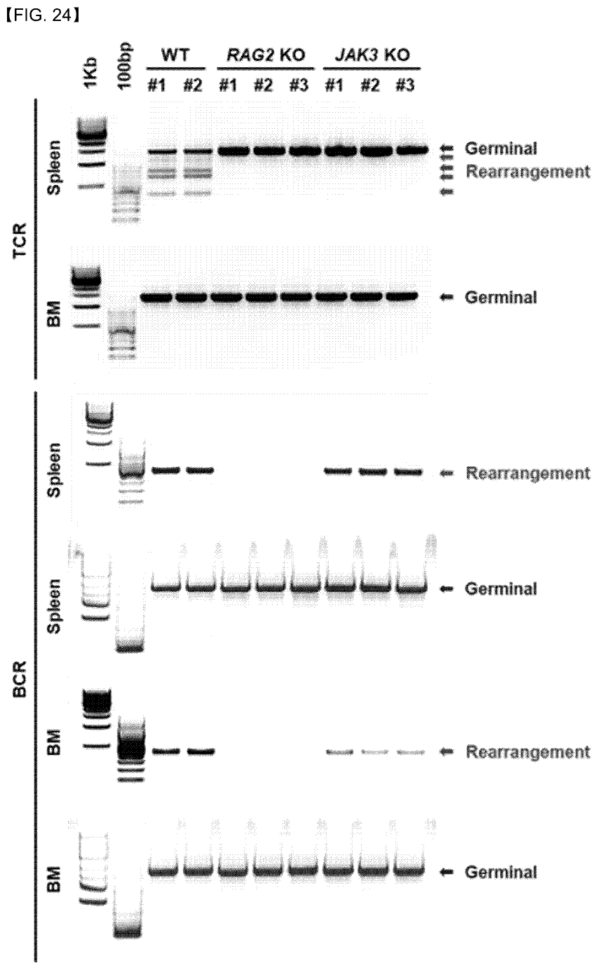

As a result, it was confirmed that no rearrangement was formed in both T and B cells in the RAG2 KO mini-pigs, whereas no rearrangement is formed in T cells, but a slight rearrangement was formed in B cells, in the JAK3 KO mini-pigs (FIGS. 23 and 24). Through these results, it was confirmed that RAG2 KO mini-pigs were T−, B−, and NK+ as previously known, but JAK3 KO mini-pigs were T−, B low, and NK−.

### Experimental Example 4: Phenotypic Analysis of JAK3 Knock-out Transgenic Cloned Mini-Pigs (2)

### Experimental Example 4-1. Survival Rate Analysis of JAK3 Knock-out Transgenic Cloned Min-Pigs

As described above, since it is determined that JAK3 knock-out causes immune function abnormalities, which affects survival, the survival rate of the JAK3 knock-out transgenic cloned mini-pigs (JAK3 KO) of Example 1 of the present disclosure raised in the SPF facility was examined.

Specifically, the survival rates of normal individuals in which JAK3 was not knocked out (wild type; WT), RAG2 knock-out transgenic cloned mini-pigs (RAG2 knock out; RAG2 KO), and the JAK3 knock-out transgenic cloned mini-pigs of Example 1 (JAK3 knock-out) out; JAK3 KO) were compared.

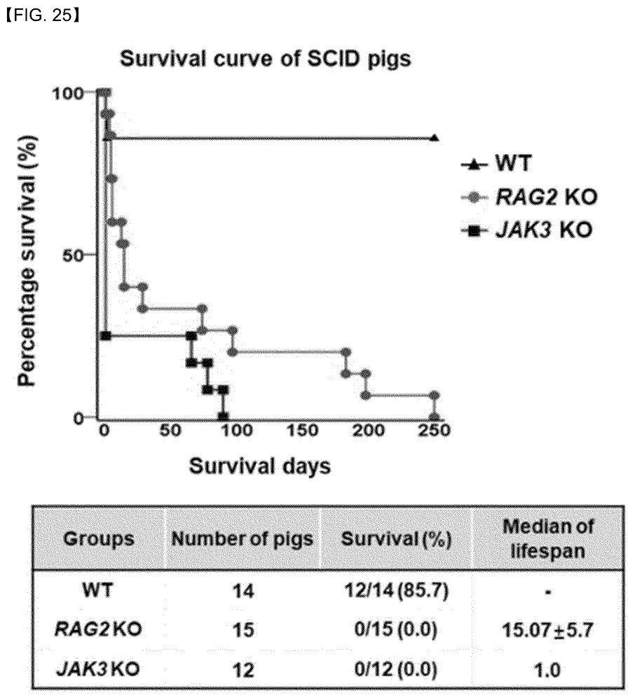

As a result, it was confirmed that the survival rate of the JAK3 KO mini-pigs was significantly lower than that of the RAG2 KO mini-pigs (Table 9 and FIG. 25).

TABLE 9
Survival rate of SCID cloned pigs

| | | JAK3 KO | | | | | | RAG2 KO | | | |
|---|---|---|---|---|---|---|---|---|---|---|---|
| | | Surrogate mother | | Surrogate mother | | Surrogate mother | | Surrogate mother | | Surrogate mother | |
| WT | | MEDI-12 | | MEDI-21 | | MEDI-26 | | GM-047 | | MEDI-14 | |
| ID | Lifespan | ID | Lifespan | ID | Lifespan | ID | Lifespan | ID | Lifespan | ID | Lifespan |
| #1 | 2 | #1 | 1 | #1 | 1 | #1 | 1 | #1 | 74 | #1 | 250 |
| #2 | 250 | #2 | 1 | #2 | 66 | #2 | 1 | #2 | 29 | #2 | 15 |
| #3 | 250 | #3 | 1 | #3 | 78 | | | #3 | 5 | #3 | 198 |
| #5 | 1 | #4 | 1 | #4 | 90 | | | #4 | 15 | #4 | 183 |
| #6 | 250 | #5 | 1 | | | | | #5 | 6 | | |
| #8 | 250 | #6 | 1 | | | | | #6 | 97 | | |
| #9 | 250 | | | | | | | #7 | 6 | | |
| #11 | 250 | | | | | | | #8 | 4 | | |
| #12 | 250 | | | | | | | #9 | 13 | | |
| #13 | 250 | | | | | | | #10 | 5 | | |
| #14 | 250 | | | | | | | #11 | 1 | | |
| #16 | 250 | | | | | | | | | | |
| #17 | 250 | | | | | | | | | | |
| #18 | 250 | | | | | | | | | | |

### Experimental Example 4-2. Confirmation of Cancer Cell Growth in JAK3 Knock-out Transgenic Cloned Mini-Pigs

As described above, since JAK3 knock-out causes immune function abnormalities, it was confirmed whether there was an immune response when foreign cells, particularly cancer, were introduced.

Specifically, for tumorigenicity analysis, 1-10×10⁵ tumor cells of a human melanoma cell line (LOX-IMVI) were injected into the right and left subcutaneous regions of WT and SCID pigs using a 1 ml syringe. After injection, tumors were collected after euthanasia on day 16 when tumor formation was observed.

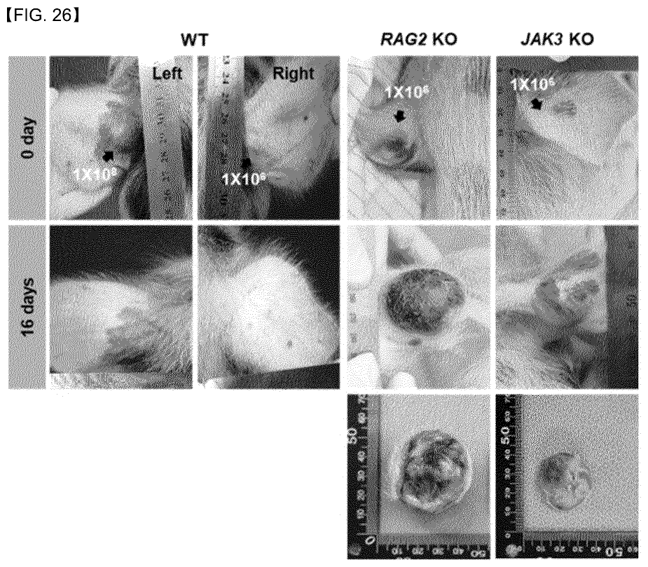

As a result, it was confirmed that the externally injected cancer cells can grow in both the JAK3 KO mini-pigs and the RAG2 KO mini-pigs because SCID has immune function abnormalities. Meanwhile, it was confirmed that no cancer cell growth was observed in WT (FIG. 26).

### Experimental Example 4-3. Confirmation of Introduction of Immune Cells into Cancer Cells and Tissue Transplanted into JAK3 Knock-out Transgenic Cloned Mini-Pigs-Immunohistochemical Staining and RT-PCR

The introduction of T, B, and NK immune cells into the tumor tissue formed by cancer cells transplanted into the animal model of Experimental Example 4-2 was examined by immunohistochemical staining and RT-PCR.

Specifically, some of the tumors collected using the antibodies shown in Table 11 were fixed in 4% (w/v) formaldehyde and embedded in paraffin, and histochemical analysis and immunohistochemical analysis were performed on 5 μm sections of glass slides to identify various types of tissue of the tumors. Additionally, to confirm that tumors were formed by the transplanted cancer cells, a PCNA antibody that reacts to proliferating cells was used for verification.

mRNA was extracted from some of the tumors for gene expression and amplified by qRT-PCR for T, B and NK cell markers using the primers shown in Table 12, to confirm that porcine T, B and NK cells were introduced into tumors in pigs injected with human cells.

TABLE 11

| | Target | | | | |
|---|---|---|---|---|---|
| Antigen | population | Clone | Isotype | Dilution | Suppler |
| CD3 | Pan-T-cells | SP7 | Rabbit IgG | 1:200 | Abcam |
| CD20 | B lymphocytes | EP459Y | Rabbit IgG | 1:250 | Abcam |
| CD335 | NKp46 | VIV-KM1 | Mouse IgG1 | 1:200 | Invitrogen |
| PCNA | Proliferating cells | PC10 | Mouse IgG2b | 1:250 | Dako |
| IgA | Plasma cells | | Goat IgA | 1:50 | BETHYL |

TABLE 12

| Gene Name | Accession No. | Primer sequence (5′→3′) | Product size |
|---|---|---|---|
| TBX212 | NM_001315722.1 | F: CCTGTACGTCCACCCAGATT | 189 bp |
| | | R: TCTGGCTCACCATCATTGAC | |
| EOMES | XM_005669315.3 | F: ACTCCATGTACACCGCTTCC | 109 bp |
| | | R: AAGAAGGACTGAACGCCGTA | |
| NKG2D (KLRK1) | NM_213813.2 | F: TGTGGAGAAAACCCATCTCC | 130 bp |
| | | R: TTGGAGCCTCTTGGTTGAAT | |
| NKp46 (NCR1) | NM_001123143.1 | F: CTCCTGGTCAAAGGAGATGG | 105 bp |
| | | R: GATCGGGGTGTCTACGGTTA | |
| NKp44 (NCR2) | XM_021098670.1 | F: AGTCCGTGAGGTTCCATCTG | 160 bp |
| | | R: AGGGTGGAGTTTTCTTGCTG | |
| GAPDH | NM_001206359.1 | F: CCCTGAGACACGATGGTGAA | 127 bp |
| | | R: GGAGGTCAATGAAGGGGTCA | |

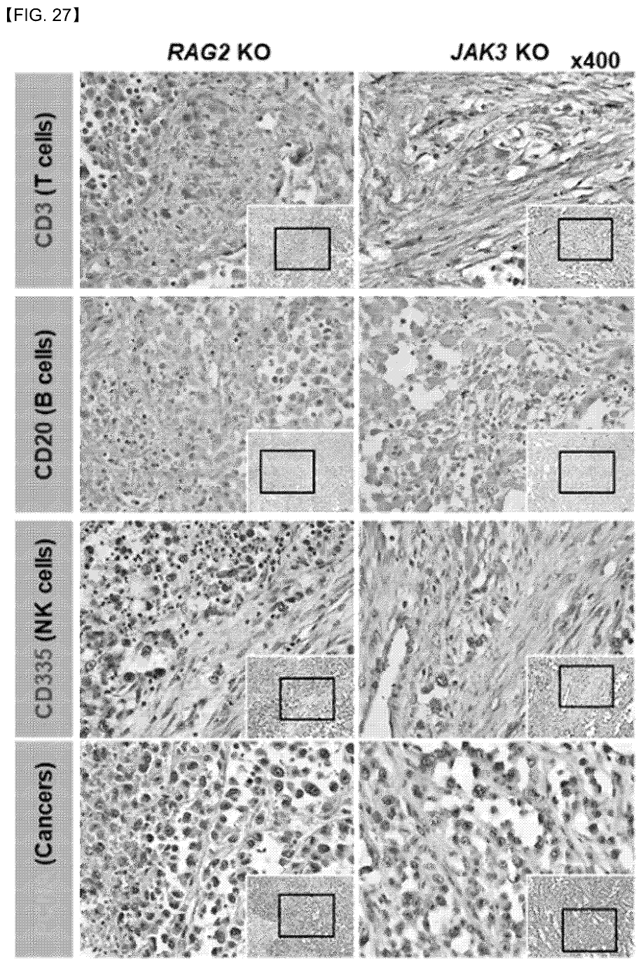

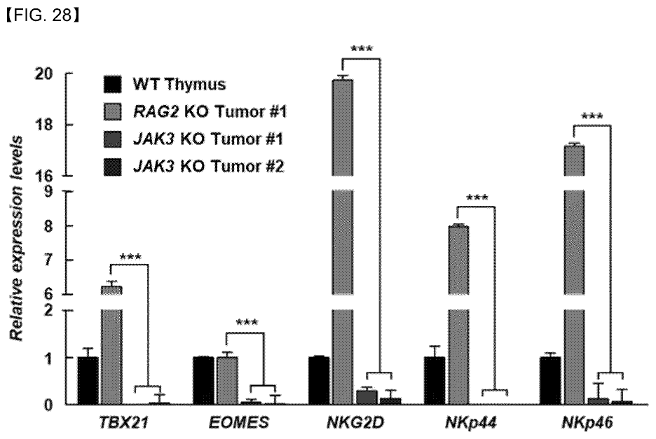

As a result, it was confirmed that NK cells were introduced into cancer cells in the RAG2 KO mini-pigs by immunohistochemical staining, and some B cells were introduced into the JAK3 KO mini-pigs (FIG. 27). As a result of examining the marker genes of NK cells by RT-PCR, the marker genes of NK cells were confirmed in the RAG2 KO mini-pigs compared to the JAK3 KO mini-pigs (FIG. 28).

From these results, it can be seen that the JAK3 knock-out mini-pigs of the present disclosure have reduced immune function and can be used also in cancer research.

### Experimental Example 5: Phenotypic Analysis of JAK3 Knock-out Transgenic Cloned Mini-Pigs (3)

### Experimental Example 5-1. Blood Analysis of JAK3 Knock-out Transgenic Cloned Min-Pigs

Blood (Lymphocyte, Monocyte and Platelet) of the JAK3 knock-out transgenic cloned mini-pigs was examined.

Specifically, for blood analysis, the number of 6 types of leukocyte-based blood cells (leukocytes, neutrophils, lymphocytes, monocytes, eosinophils, and basophils), the number of 7 types of erythrocyte-based blood cells (erythrocytes, hemoglobin, hematocrit, mean hematocrit, mean red blood cell hemoglobin, average red blood cell hemoglobin concentration, and red blood cell distribution width), the number of two types of platelet-based blood cells, and the like were measured using an automated hemocytometer HEMAVET system (Cat #HV 950, FS, Drew Scientific, Miami Lakes, FL, USA).

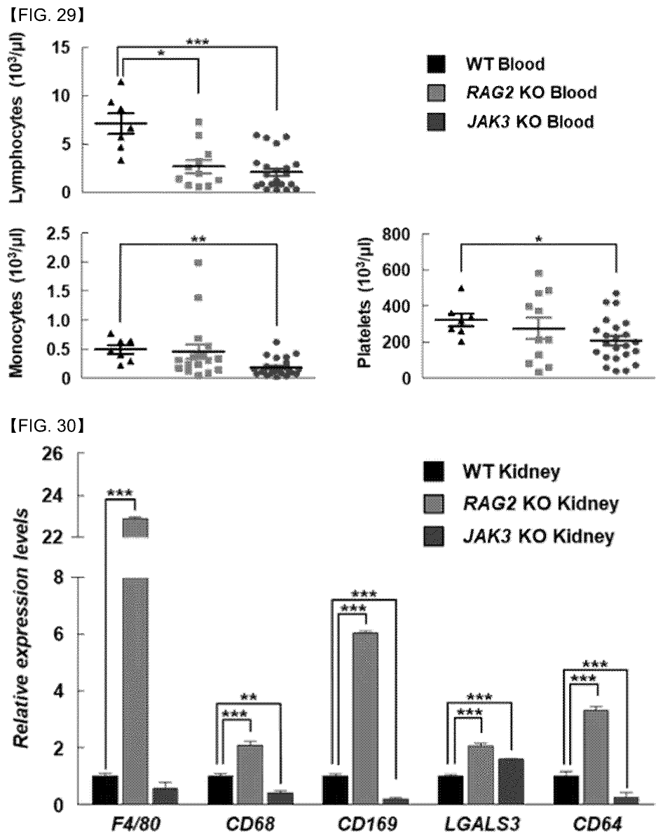

As a result, it was confirmed that decreases in lymphocytes as well as monocytes and platelets were observed in the JAK3 KO mini-pigs, and there was no difference in other hematological indicators including red blood cells (FIG. 29).

### Experimental Example 5-2. Confirmation of Macrophage-Related Gene Expression in Kidney Tissue of JAK3 Knock-out Transgenic Cloned Mini-Pigs (1)

The expression of macrophage-related genes in kidney tissues of the JAK3 knock-out transgenic cloned mini-pigs was examined.

Specifically, RT-PCR was performed using the primers shown in Table 13.

TABLE 13

| Gene Name | Accession No. | Primer sequence (5′→3′) | Product size |
|---|---|---|---|
| F4/80 (ADGRE) | XM_021083951.1 | F: CCTTCTCTTTTGGGGGTGTT | 112 bp |
| | | R: CGGACACATGGTGGTATCTG | |
| CD68 | NM_001291776.1 | F: TGGGGCATCTCTGTACTGAAC | 126 bp |
| | | R: CTGTGGCTGCTGCTTGAATC | |
| CD169 | XM_021077303.1 | F: GTGCAGTATGCCCCCAAG | 119 bp |
| | | R: GAACTGACCTGAGGGTTGCT | |
| LGALS3 | NM_001097501.2 | F: GGTTTTTCGCTTAACGATGC | 116 bp |
| | | R: GGATAGGAAGCCCCTGGATA | |
| CD64 | NM_001033011.1 | F: AATTCTGCTCCTTTGCGTTC | 127 bp |
| | | R: ATGGGGTCCCTCACATTGTA | |
| GAPDH | NM_001206359.1 | F: CCCTGAGACACGATGGTGAA | 127 bp |
| | | R: GGAGGTCAATGAAGGGGTCA | |

For the ELISA analysis method, CD169-expressing macrophages were evaluated using a porcine CD169/SIGLEC1 kit (Cat #MBS9331706, MyBioSource) by an analysis method provided by the manufacturer. Samples were diluted in DPBS/1°/0 BSA as needed and plates were read within 20 minutes at a wavelength of 450 nm using a Molecular Devices VersaMax Absorbance Microplate Reader (Molecular devices, San Jose, CA, USA). For evaluation of the results, data were analyzed using SoftMax Pro Software (Molecular Devices).

As a result of RT-PCR measurement of genes related to macrophages in kidney tissue, it was confirmed that the expression of F4/80, CD68, CD169, LGALS3 and CD64 was significantly increased in RAG2 KO mini-pigs than in WT, the expression of F4/80, CD68, CD169, LGALS3, and CD64 was decreased in JAK3 KO mini-pigs than in RAG2 KO mini-pigs, and the expression of CD68, CD169, LGALS3, and CD64 was decreased in JAK3 KO mini-pigs than in WT (FIG. 30).

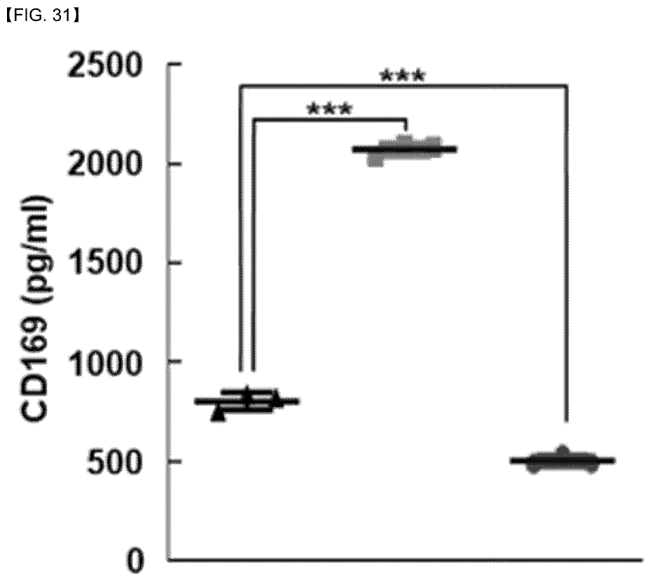

In addition, as a result of measuring CD169 related to macrophages in kidney tissue by ELISA, similar to the decreased results in RT-PCR, CD169 expression was significantly increased in RAG2 KO mini-pigs than in WT, but was decreased in JAK3 KO mini-pigs than in WT and RAG2 KO mini-pigs (FIG. 31).

### Experimental Example 5-3. Confirmation of Gene Expression in JAK3 Knock-out Transgenic Cloned Mini-Pigs (2)

The expression of CD68 among the macrophage-related genes in the kidney tissue of JAK3 knock-out transgenic cloned mini-pigs was examined.

Specifically, immunohistochemical staining was performed using the antibodies shown in Table 14, and then cells positively stained for CD68 (CD68+) through DAB (3,3′ reagent) were colored brown, and the CD68+ cells were then counted by hand and quantified.

TABLE 14

| | Target | | | | |
|---|---|---|---|---|---|
| Antigen | population | Clone | Isotype | Dilution | Suppler |
| CD68 | Monocyte/ | MAC387 | Mouse IgG1 | 1:100 | LSBio |
| | Macrophage | | | | |
| IgA | Plasma cells | | Goat IgA | 1:500 | BETHYL |

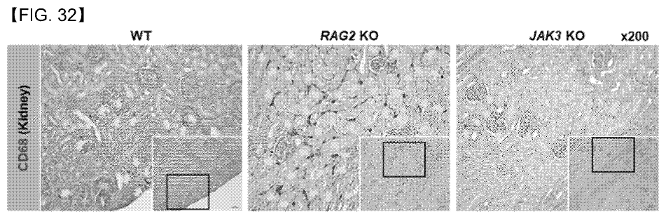

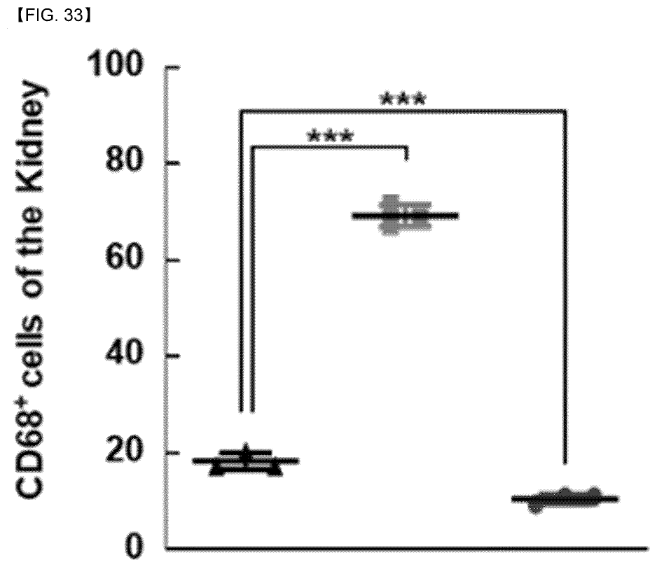

As a result, similar to the results of Experimental Example 5-2 above, it was confirmed that the number of CD68+ cells was significantly increased in RAG2 KO mini-pigs than in WT, and the number of CD68+ cells was significantly decreased in JAK3 KO mini-pigs than in WT and RAG2 KO mini-pigs (see yellow arrow+staining of FIG. 32 and see FIG. 33).

### Experimental Example 5-4. Confirmation of Gene Expression in JAK3 Knock-out Transgenic Cloned Mini-Pigs (3)

The expression of macrophage polarization-related genes (M1, M2) and the expression of cytokines in the kidney tissue of JAK3 knock-out transgenic cloned mini-pigs were examined.

Specifically, RT-PCR was performed using the primers shown in Table 15.

TABLE 15

| Gene Name | Accession No. | Primer sequence (5′→3′) | Product size |
|---|---|---|---|
| IL1β | NM_001302388.2 | F: CTCCTCACAGGGGACTTGAA | 148 bp |
| | | R: GGGTGGGTGTGTCATCTTTC | |
| TNFα | NM_214022.1 | F: CCCAGAAGGAAGAGTTTCCAG | 149 bp |
| | | R: ATACCCACTCTGCCATTGGA | |
| INOS (NOS2) | XM_013981166.2 | F: CCAAAGGGGATCTTGCTTG | 106 bp |
| | | R: AGCTCGTCTGGTGGGGTAG | |
| MCP-1(CCL2) | NM_214214.1 | F: TCTCCAGTCACCTGCTGCTA | 117 bp |
| | | R: ATACCCACTCTGCCATTGGA | |
| CXCL10 | NM_001008691.1 | F: TGGAACTCAAGGAATACCTCTCTC | 131 bp |
| | | R: CAACATGTGGGCAAGATTGA | |
| IL6 | NM_214399.1 | F: TTCACCTCTCCGGACAAAAC | 122 bp |
| | | R: TCTGCCAGTACCTCCTTGCT | |
| CD80 | XM_021068533.1 | F: GGTGCTGGTTGGTCTTTTTG | 137 bp |
| | | R: CCTTTTGCCAGTATATTCGGACT | |
| MRC1 (CD206) | NM_001255969.1 | F: CCTGCAGCTCTTGGACACTA | 127 bp |
| | | R: GAATTTCTGGGCCTCATTGT | |
| ARG1 | NM_214048.2 | F: CTCCTTTCTCCAAGGGTCAG | 122 bp |
| | | R: CAGGTCCCCGTAATCTTTCA | |
| MMP9 | NM_001038004.1 | F: GGTGTTAAGGAGCACGGAGA | 156 bp |
| | | R: GAAGTAGGTCGGAACCACGA | |
| CD163 | XM_021091120.1 | F: TGGTGCTACATGAAAACTCTGG | 135 bp |
| | | R: GCTCCTTGTCTTTTCCTCCA | |
| IL10 | NM_214041.1 | F: CACATGCTCCGGGAACTC | 126 bp |
| | | R: GCAACCCAGGTAACCCTTAAA | |
| GAPDH | NM_001206359.1 | F: CCCTGAGACACGATGGTGAA | 127 bp |
| | | R: GGAGGTCAATGAAGGGGTCA | |

For the ELISA analysis method, porcine cytokines such as IL1β (Cat #ARG81288 arigobio, Hsinchu City, Taiwan), IL2 (Cat #ARG81289 arigobio), IL10 (Cat #ARG81286, arigobio), IL12 (Cat #GR106161, arigobio), and CXCL10 (Cat #ARG81289 arigobio) were evaluated using a Duoset ELISA kit (purchase #MBS706897, MyBioSource) by an analysis method provided by the manufacturer. Samples were diluted in DPBS/1% BSA as needed and plates were read within 20 minutes at a wavelength of 450 nm using a Molecular Devices VersaMax Absorbance Microplate Reader (Molecular devices, San Jose, CA, USA). For evaluation of the results, data were analyzed using SoftMax Pro Software (Molecular Devices).

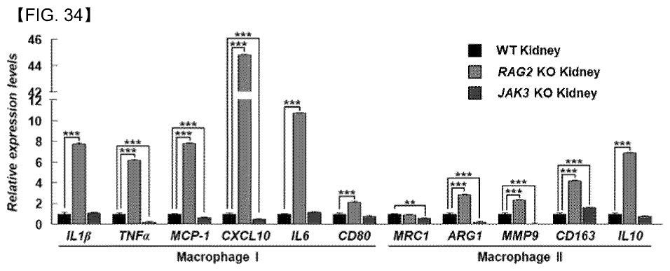

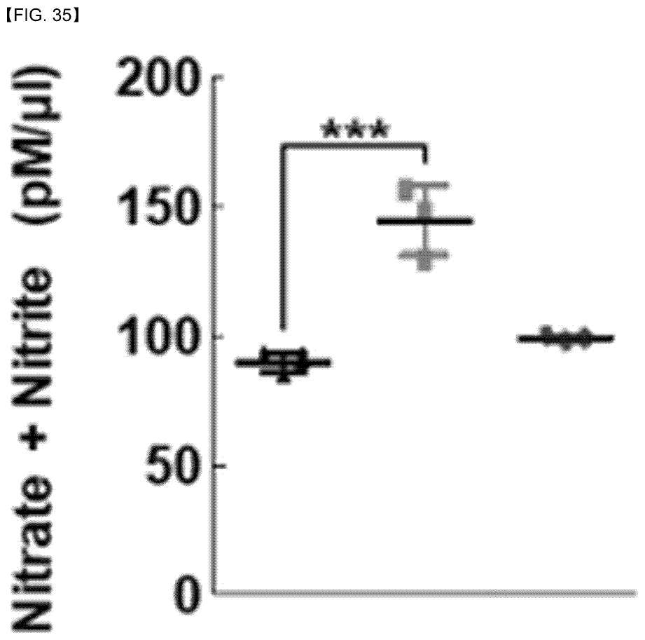

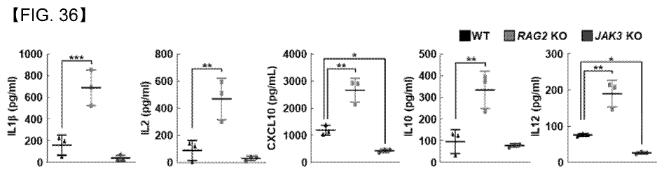

As a result of RT-PCR measurement of the macrophage polarization-related genes (M1, M2) and ELISA measurement of the cytokines, it was confirmed that the expression of M1 and M2 and the expression of the cytokines were significantly increased in RAG2 KO mini-pigs than in WT, but were decreased in JAK3 KO mini-pigs (FIGS. 34 to 36).

### Experimental Example 6: Phenotypic Analysis of JAK3 Knock-out Transgenic Cloned Mini-Pigs (4)

### Experimental Example 6-1. Confirmation of Lung/Visceral Abnormalities of JAK3 Knock-out Transgenic Cloned Min-Pigs

Lung/visceral abnormalities of the JAK3 knock-out transgenic cloned mini-pigs (JAK3 knock out; JAK3 KO) of Example 1 were compared and examined.

Specifically, normal individuals in which JAK3 was not knocked out (wild type; WT), RAG2 KO mini-pigs euthanized due to a worsening condition, and the JAK3 KO mini-pigs of Example 1 were necropsied, and inflammation in the lung/intestines was identified visually and by H&E staining and immunohistochemical staining.

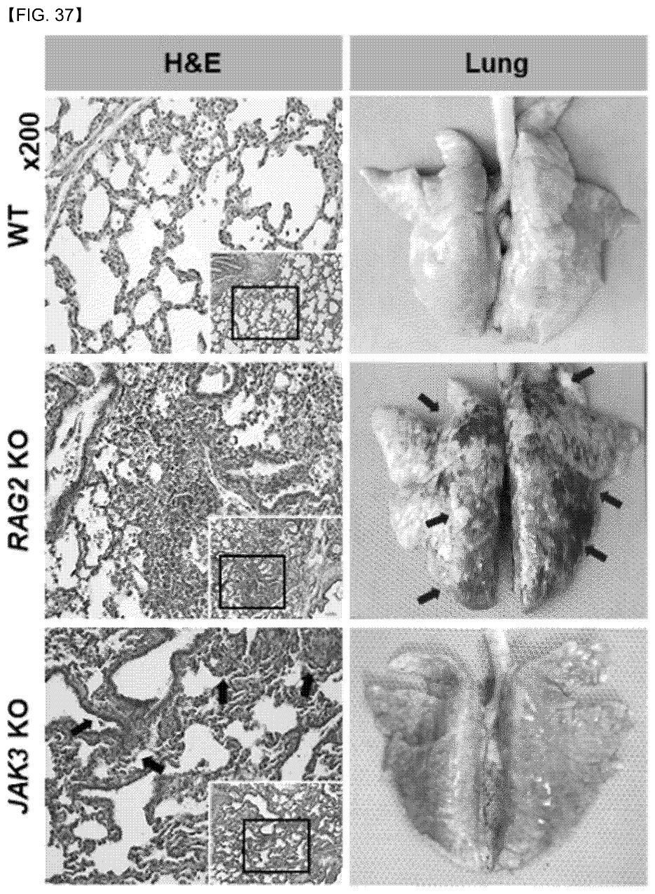

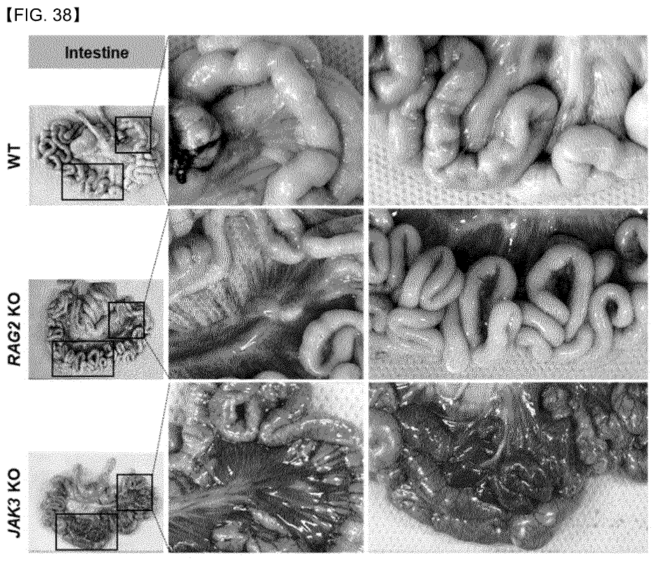

As a result, no significant lung inflammation was observed in normal individuals in which JAK3 was not knocked out (wild type; WT) and JAK3 KO mini-pigs, whereas inflammatory diseases, including pneumonia were mainly observed in RAG2 KO mini-pigs (FIG. 37, arrows in RAG2 KO lung image).

Meanwhile, no significant visceral abnormalities were found in normal individuals in which JAK3 was not knocked out (wild type; WT) and RAG2 KO mini-pigs, whereas visceral abnormalities were confirmed in JAK3 KO mini-pigs (FIG. 38).

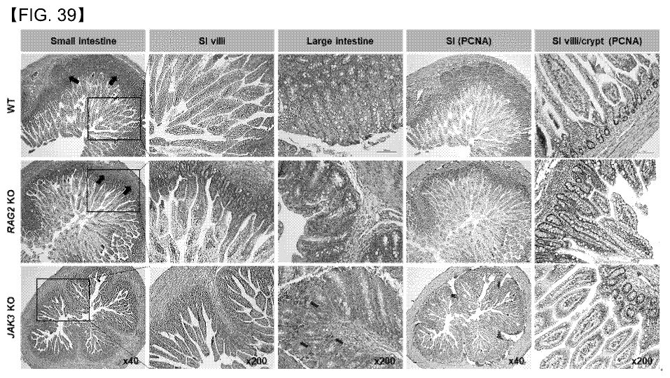

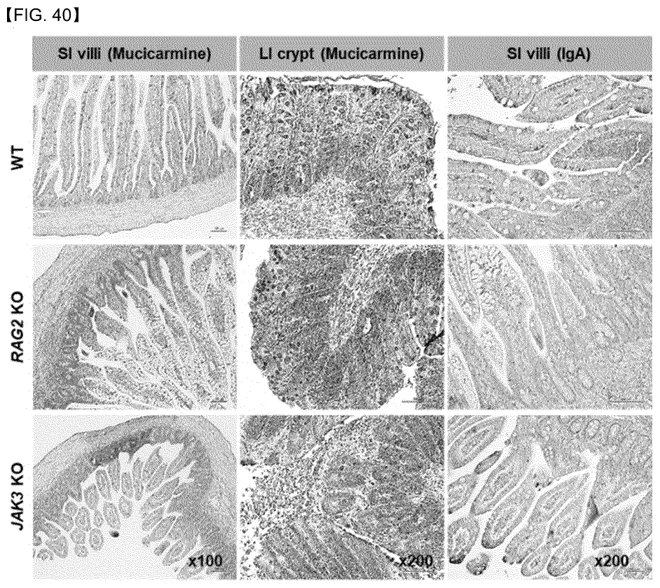

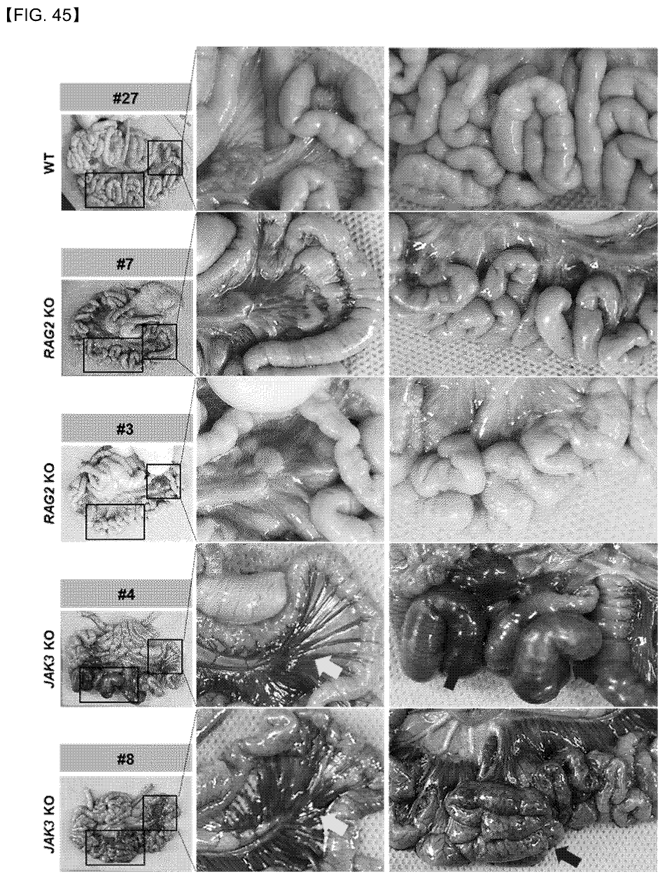

Specifically, as a result of confirmation through H&E staining and immunohistochemical staining, Peyer's patch (black arrow), which is an intestinal immune organ, was reduced in the intestines of RAG2 KO mini-pigs while some were observed, whereas Peyer's patch was not present in JAK3 KO mini-pigs at all. In addition, the length of intestinal villi was much shorter than normal, and even in immunostaining using a proliferating cell nuclear antigen (PCNA) antibody, which is stained specifically for cell proliferation, when normal, only the crypt where intestinal stem cells are present should be stained specifically, but it was confirmed that the villi were stained non-specifically in the intestines of JAK3 KO mini-pigs (FIGS. 39 and 44).

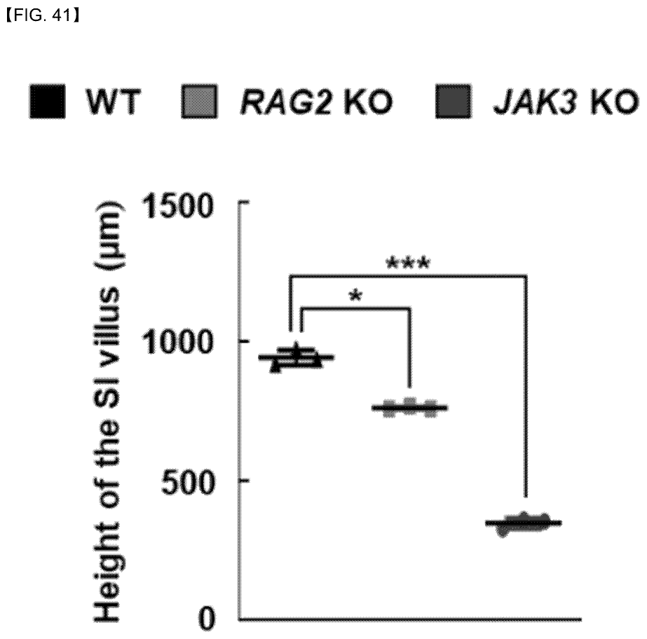

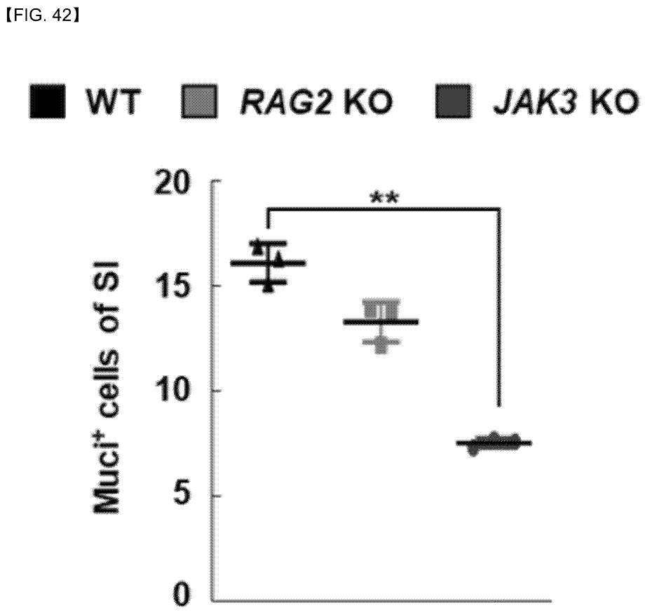

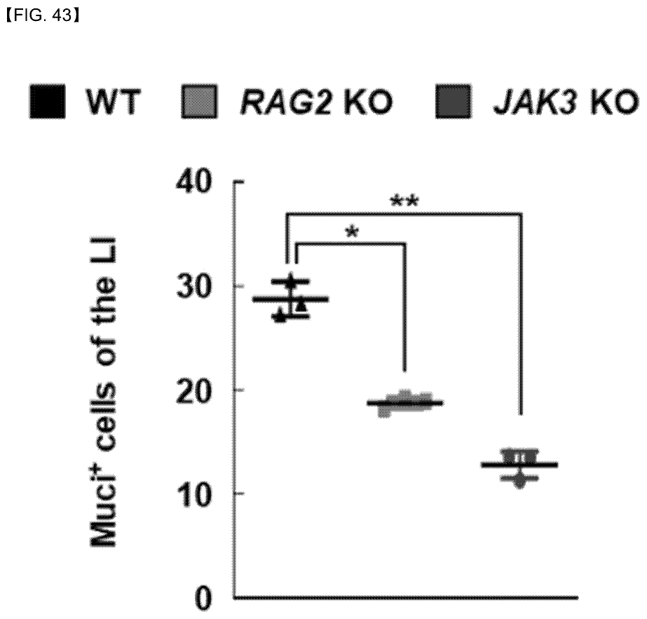

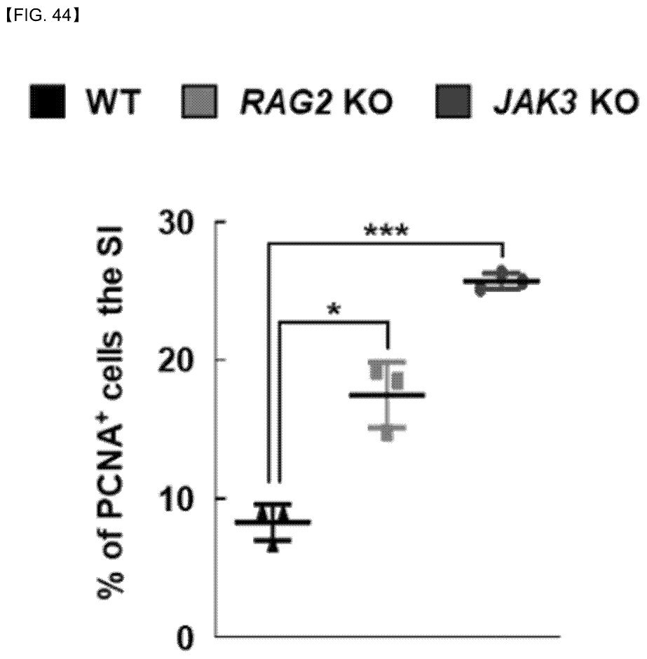

In addition, in special mucicamine staining, which specifically stains goblet cells that secrete intestinal mucus, a significantly remarkable decrease was confirmed in the intestines of JAK3 KO mini-pigs (FIGS. 40 to 43).

It was also confirmed that immunoglobulin A (IgA) secreted from intestinal plasma cells was not observed at all in the intestines of RAG2 KO mini-pigs not having B cells, whereas the intestines of JAK3 KO mini-pigs had a reduced number of IgA compared to WT and stained (FIG. 40).

These results indicate that the intestines of JAK3 KO mini-pigs are not properly differentiated.

### Experimental Example 6-2. Identification of Intestines of JAK3 Knock-out Transgenic Cloned Mini-Pigs

Visceral abnormalities of the JAK3 knock-out transgenic cloned mini-pigs (JAK3 knock out; JAK3 KO) of Example 1 were compared and examined.

Specifically, normal individuals in which JAK3 was not knocked out (wild type; WT), RAG2 KO mini-pigs that were euthanized due to a worsening condition, and the JAK3 KO mini-pigs of Example 1 were necropsied, and intestinal inflammation was identified with the naked eye.

As a result, it was confirmed that the intestines of the JAK3 KO mini-pigs showed internal bleeding (blue arrow in JAK3 KO) and congestion of mesenteric vessels (yellow arrow in JAK3 KO), and as a result of histological analysis, the mesenteric vessels lost elasticity and expanded. Since these symptoms were found in all JAK3 individuals who consumed milk powder or feed after hysterectomy, these results indicate that intestinal abnormalities occurred due to poor intestinal differentiation (FIG. 45).

The exemplary embodiments of the present disclosure have been mainly described. It will be understood by those of ordinary skill in the art to which the present disclosure pertains that the present disclosure can be embodied in a modified form without departing from the essential characteristics of the present disclosure. Thus, the above-described embodiments should be construed as being provided for illustrative purposes only and not for purposes of limitation. The scope of the present disclosure is defined by the claims rather than the foregoing description, and all differences within the equivalent scope should be construed as being included in the present disclosure.

1. A recombinant expression vector comprising:
a nucleotide sequence encoding a guide RNA (gRNA) that hybridizes to a DNA encoding a Janus kinase 3 (JAK3) gene;
a nucleotide sequence encoding a Cas9 protein; and
a promoter operably linked to the nucleotide sequence.

2. The recombinant expression vector of claim 1, wherein the gRNA consists of the nucleotide sequence of SEQ ID NO: 1 or SEQ ID NO: 2.

3. (canceled)

4. A method of constructing a severe combined immunodeficiency animal model, the method comprising:
forming a nuclear transfer embryo by transplanting a transgenic cell line into which the recombinant expression vector of claim 1 is introduced into an enucleated egg obtained from an animal other than a human; and
transferring the nuclear transfer embryo into a fallopian tube of a surrogate mother, which is an animal other than a human.

5. The method of claim 4, wherein the animal is a mini-pig.

6. The method of claim 4, wherein the severe combined immunodeficiency is caused by Janus kinase 3 (JAK3) knock-out.

7. The method of claim 6, wherein the knock-out is caused by mutating a nucleotide sequence corresponding to SEQ ID NO: 3 to any one nucleotide sequence selected from the group consisting of SEQ ID NO: 4 to SEQ ID NO: 6.

8. A severe combined immunodeficiency animal model with a Janus kinase 3 (JAK3) gene mutation.

9. The severe combined immunodeficiency animal model of claim 8, wherein the animal model is an animal model in which the JAK3 gene is knocked out.

10. The severe combined immunodeficiency animal model of claim 9, wherein the knock-out is caused by mutating a nucleotide sequence corresponding to SEQ ID NO: 3 to any one nucleotide sequence selected from the group consisting of SEQ ID NO: 4 to SEQ ID NO: 6.

11. The severe combined immunodeficiency animal model of claim 8, wherein the animal is a mini-pig.

12. The severe combined immunodeficiency animal model of claim 8, wherein the animal is used for artificial blood development, xenotransplantation, or severe immunodeficiency disease animal models.
